(19) United States
(12) Patent Application Publication
(10) Pub. No.: US 2010/0198100 A1
(43) Pub. Date: Aug. 5, 2010

(54) BIOELECTRICAL IMPEDANCE MEASUREMENT BODY ATTACHMENT UNIT AND BODY FAT MEASUREMENT DEVICE

(75) Inventors: Shojiro Oku, Kyoto (JP); Toshikazu Shiga, Shiga (JP); Yoshitake Oshima, Kyoto (JP); Toshihiko Abe, Tokyo (JP); Shuhei Fujiwara, Osaka (JP); Josuke Ebisu, Hyogo (JP); Akihisa Narai, Mie (JP)

Correspondence Address: OSHA LIANG L.L.P., TWO HOUSTON CENTER, 909 FANNIN, SUITE 3500, HOUSTON, TX, 77010, US

(73) Assignee: OMRON HEALTHCARE CO., LTD., Kyoto (JP)

(21) Appl. No.: 12/665,417

(22) PCT Filed: Jun. 5, 2008
(86) PCT No.: PCT/JP2008/060337

§ 371 Date: Jan. 12, 2010

(30) Foreign Application Priority Data
Jul. 19, 2007 (JP) 2007-188616

(51) Int. Cl. A61B 5/053

(52) U.S. Cl. 600/547

(57) ABSTRACT

A bioelectrical impedance measurement abdomen attachment unit (100A) includes an abdomen rear surface side base portion (110A), an abdomen front surface side base portion (120A), and a pair of abdomen front surface side belt members (143a, 143b). The abdomen front surface side base portion (120A) includes a sheet (122) arranged with a pair of wind-up units (130a, 130b), and an electrode film (126) arranged with an impedance measurement electrode (128). Each wind-up unit (130a, 130b) includes a wind-up mechanism for accommodating the abdomen front surface side belt members (143a, 143b) in a winding manner, where the abdomen front surface side belt member (143a, 143b) is pulled out and connected to the abdomen rear surface side base portion (110A) by way of a connection tool in the attached state. According to such a configuration, there is obtained a bioelectrical impedance measurement abdomen attachment unit which enables the electrodes to be pressed against the body of the subject with a constant load at satisfactory reproducibility and which is not painful to the subject.

### TECHNICAL FIELD

The present invention relates to a bioelectrical impedance measurement body attachment unit attached by being wrapped around a body of a subject to measure a bioelectrical impedance, and a body fat measurement device for calculating a body fat mass of the subject by measuring the bioelectrical impedance using the bioelectrical impedance measurement body attachment unit.

### BACKGROUND ART

A body fat mass is recently being given attention as one index for knowing a health condition of a subject. In particular, a visceral fat mass is given attention as an index for making determination on whether or not a visceral obesity is present. The visceral obesity is said to induce lifestyle-related diseases that easily causes arterial sclerosis such as diabetes, a high blood pressure and hyperlipemia, and the use of the above index is expected from a standpoint of preventing such diseases. In this case, the visceral fat is a fat that accumulates around internal organs on an inner side of an abdominal muscle, and is distinguished from a subcutaneous fat that accumulates on a surface layer of the abdomen. An area (hereinafter referred to as visceral fat area) occupied by the visceral fat at a cross section of the abdomen of a portion corresponding to an umbilicus position is generally adopted for the index indicating the visceral fat mass.

Normally, an image analyzing method using a tomographic image of the abdomen photographed using an X-ray CT (Computer Tomography) or an MRI (Magnetic Resonance Imaging) is used to measure the visceral fat mass. In such an image analyzing method, the visceral fat area is calculated from the acquired tomographic image of the abdomen. However, in order to use such a method, a large facility that may be installed in medical institutions such as the X-ray CT and the MRI is required, and thus the visceral fat mass is very difficult to measure on a daily basis. A problem of exposure also arises when the X-ray CT is used, and thus such a method may not necessarily be a preferable measurement method.

As a measurement method taking the place thereof, application of a bioelectrical impedance method is being reviewed. The bioelectrical impedance method is a method of measuring the body fat mass widely used in a body fat measurement device for domestic use, where electrodes are brought into contact with four limbs, and the bioelectrical impedance is measured using such electrodes to calculate the body fat mass from the measured bioelectrical impedance. The above-described body fat measurement device accurately measures a degree of accumulation of the body fat by sites of the body such as the entire body or four limbs, or the body (trunk of the body), and is widely used in households and the like.

However, the conventional body fat measurement device measures the degree of accumulation of the body fat by sites of the body such as the entire body or four limbs, or the body (trunk of the body) as described above, and does not individually extract and accurately measure the degree of accumulation of the visceral fat or the degree of accumulation and the subcutaneous fat. This is because the body includes not only the visceral fat but also the subcutaneous fat, as described above, and thus accurately measuring the visceral fat mass and the subcutaneous fat mass individually is difficult in the body fat measurement device described above.

In order to solve such problems, consideration is being made of bringing an electrode directly in contact with the body, measuring the bioelectrical impedance using the electrode, and accurately measuring the visceral fat mass and the subcutaneous fat mass individually based thereon. For instance, Japanese Unexamined Patent Publication No. 2002-369806 (Patent Document 1) discloses a body fat measurement device configured such that the electrode is arranged in contact with the body by arranging an electrode on an inner peripheral surface of a belt member and wrapping and fixing the belt member to the body. The body fat measurement device disclosed in Japanese Unexamined Patent Publication No. 2002-369806 enables highly accurate measurement of the visceral fat mass and the subcutaneous fat mass, which has been difficult in the related art, by measuring the bioelectrical impedance using the electrode arranged in contact with the body of the subject using the belt member.

Patent Document 1: Japanese Unexamined Patent Publication No. 2002-369806

### DISCLOSURE OF THE INVENTION

### Problems to be Solved by the Invention

When measuring a bioelectrical impedance using the above-described bioelectrical impedance method, measurement is carried out by bringing an electrode directly in contact with a part of a body of a subject, and thus it is important to stably maintain a pressing strength of the electrode with respect to the body surface constant for every measurement. However, this is not easy to achieve as a shape and a size of the body of the subject differ among individuals. In particular, a difference among individuals is large in the shape and the size of the body, where stably ensuring the pressing strength of the electrode with respect to the body is very difficult when arranging the electrode so as to be in contact with the body of the subject using the bioelectrical impedance measurement body attachment unit including a belt member. This is because in the body fat measurement device described in Japanese Unexamined Patent Publication No. 2002-369806, a wrapping strength of the belt member differs for every attachment as the attachment of the belt member to the body is committed to human hand, whereby the pressing strength of the electrode to the body also differs for every attachment as a result.

In the case where the pressing strength of the electrode with respect to the body surface varies, such a variation appears as a variation of a contact resistance between the electrode and a body surface, which may lower the measurement accuracy. Therefore, it is very important that the bioelectrical impedance measurement body attachment unit is configured such that the electrode is always stably pressed against the body of the subject with a constant load regardless of the subject for every measurement.

On the other hand, in the case where the belt member is strongly wrapped around the body of the subject to ensure the pressing strength of the electrode with respect to the body, the body of the subject is tightened by the belt member, which may be painful to the subject. In particular, as the shape of the body (in particular, abdomen of the body) fluctuates with breathing motion (normally, a body peripheral length increases in inhaling motion and the body peripheral length reduces in exhaling motion), the user may feel a strong oppressing feeling in the inhaling motion, which may force great pain on the subject.

In the case where the bioelectrical impedance is measured with the electrode in contact with the body of the subject, a value of the measured bioelectrical impedance is known to fluctuate with the breathing motion of the subject. Major factors thereof being that the shape of the body changes with the breathing motion and a body composition between the electrodes arranged in contact with the body fluctuates, that the distance between the electrodes fluctuates with change in the shape of the body, that the contacting state of the electrode and the body surface fluctuates and the contact resistance changes, or the like. The fluctuation in the value of the bioelectrical impedance involved in such breathing motion inhibits the high accuracy measurement of a visceral fat mass and a subcutaneous fat mass, whereby some kind of measures needs to be taken.

In view of solving the above problems, it is an object of the present invention to provide a bioelectrical impedance measurement body attachment unit that enables the electrodes to be pressed against the body of the subject at satisfactory reproducibility with a constant load in the attachment state and that does not give pain to the subject, and a body fat measurement device equipped with the same, and also to provide a body fat measurement device capable of detecting the breathing state of the subject at high accuracy and measuring the body fat mass, in particular, the visceral fat mass and the subcutaneous fat mass at high accuracy. Furthermore, it is also an object of the present invention to provide a bioelectrical impedance measurement body attachment unit enabling the attachment task to be very easily performed, and a body fat measurement device equipped with the same.

### Means for Solving the Problems

In accordance with one aspect of the present invention, a bioelectrical impedance measurement body attachment unit based on the present invention is attached to a body of a subject to measure a bioelectrical impedance, and includes a first base portion, a second base portion, a first coupling mechanism, a second coupling mechanism, and a plurality of impedance measurement electrodes. The first base portion is placed on one of a body front surface or a body rear surface of the subject in an attached state; and the second base portion is placed on the other one, different from the one above, of the body front surface or the body rear surface of the subject in the attached state. The first coupling mechanism couples a portion closer to one end of the first base portion and a portion closer to one end of the second base portion in the attached state; and the second coupling mechanism couples a portion closer to the other of the first base portion and a portion closer to the other of the second base portion in the attached state. The plurality of impedance measurement electrodes are arranged on at least either one of the first base portion or the second base portion and brought into contact with a body surface of the subject in the attached state. The first coupling mechanism includes a first belt member and a first biasing portion. The first belt member has one end attached to the portion closer to the one end of the first base portion and the other end removably attached to the portion closer to the one end of the second base portion. The first biasing portion biases the first belt member towards the portion closer to the one end of the first base portion. The second coupling mechanism includes a second belt member and a second biasing portion. The second belt member has one end attached to the portion closer to the other end of the first base portion and the other end removably attached to the portion closer to the other end of the second base portion. The second biasing portion biases the second belt member towards the portion closer to the other end of the first base portion.

According to such a configuration, the first belt member and the second belt member are constantly pulled by the first biasing portion and the second biasing portion in the attached state. Thus, the body of the subject is tightened with a substantially constant tightening strength by the bioelectrical impedance measurement body attachment unit based on the biasing force of the first biasing portion and the second biasing portion, and the electrodes can be pressed against the body of the subject with a substantially constant load. Furthermore, according to the above-described configuration, the bioelectrical impedance measurement body attachment unit is configured by being divided to the first base portion and the second base portion, and the first base portion and the second base portion are coupled by a pair of first coupling mechanism and second coupling mechanism, whereby the bioelectrical impedance measurement body attachment unit can be very easily attached to the body of the subject. Furthermore, with the above-described configuration, a wrapping length of the bioelectrical impedance measurement body attachment unit changes following the breathing motion of the subject by appropriately adjusting the biasing force of the biasing portion, and thus the subject does not feel an excessive oppressing feeling, and a bioelectrical impedance measurement body attachment unit that is not painful to the subject can be obtained.

In the bioelectrical impedance measurement body attachment unit according to the present invention, one of the first base portion or the second base portion is preferably configured by a sheet-like member deformable so as to lie along a shape of the body surface of the subject in the attached state; and the impedance measurement electrode is desirably arranged on a main surface on a side facing the body of the subject of the sheet-like member.

According to such a configuration, the impedance measurement electrode can be easily arranged in contact with the body surface of the subject by bringing the sheet-like member into contact with the body of the subject, whereby a bioelectrical impedance measurement body attachment unit excelling in handleability can be obtained.

In the bioelectrical impedance measurement body attachment unit according to the present invention, one of the first base portion or the second base portion preferably includes a first sheet-like member deformable so as to lie along a shape of the body surface of the subject in the attached state, and a second sheet-like member, which is a part different from the first sheet-like member, deformable so as to lie along a shape of the body surface by being interposed between the body of the subject and the first sheet-like member in the attached state; and in this case, the impedance measurement electrode is desirably arranged on a main surface on a side facing the body of the subject of the second sheet-like member.

According to such a configuration, the second sheet-like member is brought into contact with the body of the subject, and the first sheet-like member is attached from above the second sheet-like member, so that the impedance measurement electrode can be easily arranged in contact with the body surface of the subject. Therefore, a bioelectrical impedance measurement body attachment unit excelling in handleability can be obtained. According to such a configuration, excellent hygienic aspect is realized since the second sheet-like member including the impedance measurement electrode is used as a biological electrode sheet of disposable type.

In the bioelectrical impedance measurement body attachment unit according to the present invention, the base portion placed on the body front surface of the subject in the attached state of the first base portion and the second base portion preferably includes a mark for alignment with an umbilicus position of the subject.

According to such a configuration, the impedance measurement electrode can be accurately positioned and attached to the body surface of the subject.

In the bioelectrical impedance measurement body attachment unit according to the present invention, the first base portion preferably includes an interval adjustment mechanism for adjusting an interval between the portion closer to the one end attached with the first coupling mechanism and the portion closer to the other end attached with the second coupling mechanism to an arbitrary distance.

According to such a configuration, since the interval between the portion closer to one end of the first base portion attached with the first coupling mechanism and the portion closer to the other end of the first base portion attached with the second coupling mechanism can be adjusted using an interval adjustment mechanism according to the body lateral width of the subject, the bioelectrical impedance measurement body attachment unit can be more reliably fitted to the body of the subject without a gap.

In the bioelectrical impedance measurement body attachment unit according to the present invention, the first base portion preferably includes a first wind-up mechanism for accommodating the first belt member in a winding manner, and a second wind-up mechanism for accommodating the second belt member in a winding manner; and in this case, the first biasing portion is desirably arranged in the first wind-up mechanism; and the second biasing portion is desirably arranged in the second wind-up mechanism.

According to such a configuration, the belt member is wound up and pulled out using the wind-up mechanism including the biasing portion, whereby a bioelectrical impedance measurement body attachment unit excelling in handleability can be obtained.

In accordance with another aspect of the present invention, a body fat measurement device according to the present invention includes: the bioelectrical impedance measurement body attachment unit according to any one of the above; an impedance measuring portion for measuring a bioelectrical impedance of the subject using the plurality of impedance measurement electrodes; and a body fat mass calculating portion for calculating a body fat mass of the subject based on the bioelectrical impedance measured by the impedance measuring portion.

According to such a configuration, there is obtained a body fat measurement device including a bioelectrical impedance measurement body attachment unit which enables the electrodes to be pressed against the body of the subject with a substantially constant load with satisfactory reproducibility in the attached state and which is not painful to the subject. Therefore, a body fat measurement device capable of calculating the body fat mass at high accuracy can be obtained.

In a body fat measurement device based on the present invention, when the first base portion includes the first wind-up mechanism and the second wind-up mechanism, the body fat measurement device preferably further includes a body peripheral length measurement unit for measuring a body peripheral length of the subject by detecting a pulled-out amount of the first belt member from the first wind-up mechanism and a pulled-out amount of the second belt member from the second wind-up mechanism; and a body fat mass calculating portion for calculating a body fat mass of the subject based on the bioelectrical impedance measured by the impedance measuring portion and the body peripheral length of the subject measured by the body peripheral length measurement unit.

According to such a configuration, the body peripheral length of the subject can be easily and automatically measured by attaching the bioelectrical impedance measurement body attachment unit, and the body fat can be measured at high accuracy by calculating the body fat mass using the obtained body peripheral length.

In the body fat measurement device according to the present invention, the body fat measurement device preferably further includes: a breathing state detecting portion for measuring a fluctuation of the body peripheral length of the subject detected by the body peripheral length measurement unit and detecting a breathing state of the subject based on the measured fluctuation of the body peripheral length of the subject with the bioelectrical impedance measurement body attachment unit attached to the body of the subject; wherein the body fat mass calculating portion desirably calculates the body fat mass of the subject based on information of the bioelectrical impedance measured by the impedance measuring portion, the body peripheral length of the subject measured by the body peripheral length measurement unit, and the breathing state detected by the breathing state detecting portion.

According to such a configuration, the breathing state of the subject can be detected at high accuracy with a simple configuration of detecting the fluctuation of the wrapping length of the bioelectrical impedance measurement body attachment unit during the measurement. Through the use of such a detection method, the change in the body peripheral length of the subject involved in the breathing motion can be captured at high accuracy, and thus a body fat measurement device capable of calculating the body fat mass at high accuracy can be obtained.

In the body fat measurement device according to the present invention, the body fat mass calculating portion preferably extracts a bioelectrical impedance measured at a timing of transitioning from an exhaling motion to an inhaling motion detected by the breathing state detecting portion from time-series data of the bioelectrical impedance measured by the impedance measuring portion and calculates the body fat mass of the subject from the extracted bioelectrical impedance.

According to such a configuration, the bioelectrical impedance can be measured excluding an influence of the fluctuation of the bioelectrical impedance that occurs with the breathing motion, and thus the body fat mass can be calculated at high accuracy.

In the body fat measurement device according to the present invention, the body fat mass calculating portion preferably includes a visceral fat mass calculating part for calculating a visceral fat mass of the subject.

The bioelectrical impedance needs to be measured with the electrodes arranged in contact with the body of the subject in order to measure the visceral fat mass at high accuracy, and thus the visceral fat mass can be particularly calculated at high accuracy with the body fat measurement device of the above configuration.

In the body fat measurement device according to the present invention, the body fat mass calculating portion preferably includes a subcutaneous fat mass calculating part for calculating a subcutaneous fat mass at an abdomen of the subject.

The bioelectrical impedance needs to be measured with the electrodes arranged in contact with the body of the subject in order to measure the subcutaneous fat mass at the abdomen at high accuracy, and thus the subcutaneous fat mass at the abdomen can be particularly calculated at high accuracy with the body fat measurement device of the above configuration.

### EFFECTS OF THE INVENTION

According to the present invention, a bioelectrical impedance measurement body attachment unit which enables electrodes to be pressed against a body of a subject with a constant load in an attached state with satisfactory reproducibility and which is not painful to the subject, and a body fat measurement device equipped with the same are provided, and furthermore, a body fat measurement device capable of detecting a breathing state of the subject at high accuracy and capable of measuring a body fat mass, particularly, a visceral fat mass and a subcutaneous fat mass at high accuracy is provided. Furthermore, according to the present invention, the bioelectrical impedance measurement body attachment unit enabling the attachment task to be very easily performed, and the body fat measurement device equipped with the same are provided.

### BRIEF DESCRIPTION OF THE DRAWINGS

FIG. 1 is a view showing function blocks of a body fat measurement device according to a first embodiment of the present invention.

FIG. 2 is a flowchart showing operation procedures of the body fat measurement device in measuring a visceral fat area, a subcutaneous fat area, and a body fat percentage using the body fat measurement device according to the first embodiment of the present invention.

FIG. 3 is a schematic perspective view showing an outer appearance structure of a body fat measurement device according to the first embodiment of the present invention.

FIG. 4A is a top view of an abdomen rear surface side base portion of a bioelectrical impedance measurement abdomen attachment unit shown in FIG. 3.

FIG. 4B is a side view of the abdomen rear surface side base portion of the bioelectrical impedance measurement abdomen attachment unit shown in FIG. 3.

FIG. 5A is a top view of an abdomen front surface side base portion of the bioelectrical impedance measurement abdomen attachment unit shown in FIG. 3.

FIG. 5B is a side view of a portion excluding an electrode film of the abdomen front surface side base portion of the bioelectrical impedance measurement abdomen attachment unit shown in FIG. 3.

FIG. 6A is a bottom view of the electrode film of the abdomen front surface side base portion of the bioelectrical impedance measurement abdomen attachment unit shown in FIG. 3.

FIG. 6B is a side view of the electrode film of the abdomen front surface side base portion of the bioelectrical impedance measurement abdomen attachment unit shown in FIG. 3.

FIG. 7 is a cross-sectional view of the abdomen front surface side base portion of the bioelectrical impedance measurement abdomen attachment unit shown in FIG. 3.

FIG. 8 is a perspective view of a wind-up mechanism shown in FIG. 7.

FIG. 9 is a schematic cross-sectional view showing a state where the bioelectrical impedance measurement abdomen attachment unit according to the first embodiment of the present invention is attached to the abdomen of the subject.

FIG. 10 is a side view of the abdomen front surface side base portion of the bioelectrical impedance measurement body attachment unit according to a first variant of the first embodiment of the present invention.

FIG. 11 is a schematic cross-sectional view showing a state in which the bioelectrical impedance measurement body attachment unit according to the first variant of the first embodiment of the present invention is attached to the abdomen of the subject.

FIG. 12A is a top view of the abdomen rear surface side base portion of the bioelectrical impedance measurement body attachment unit according to a second variant of the embodiment of the present invention.

FIG. 12B is a side view of the abdomen rear surface side base portion of the bioelectrical impedance measurement body attachment unit according to the second variant of the embodiment of the present invention.

FIG. 13 is a schematic cross-sectional view showing a state in which the bioelectrical impedance measurement body attachment unit according to the second variant of the first embodiment of the present invention is attached to the abdomen of the subject.

FIG. 14 is a perspective view of the abdomen front surface side base portion of the bioelectrical impedance measurement body attachment unit according to a third variant of the first embodiment of the present invention.

FIG. 15 is a view showing function blocks of a body fat measurement device according to a second embodiment of the present invention.

FIG. 16 is a schematic cross-sectional view of a portion excluding the electrode film of the abdomen side base portion of the bioelectrical impedance measurement body attachment unit equipped in the body fat measurement device according to the second embodiment of the present invention.

FIG. 17 is a perspective view of a wind-up mechanism shown in FIG. 16.

FIG. 18 is a schematic view describing a configuration of a body peripheral length measurement unit of the body fat measurement device according to a third embodiment of the present invention and a mechanism for automatically measuring the body peripheral length of the subject with the body peripheral length measurement unit.

FIG. 19 is a flowchart showing the operation procedures of the body fat measurement device in measuring the visceral fat area, the subcutaneous fat area, and the body fat percentage using the body fat measurement device according to the second embodiment of the present invention.

FIG. 20 is a view showing function blocks of a body fat measurement device according to a third embodiment of the present invention.

FIG. 21 is a graph showing a relationship of a fluctuation of the body peripheral length of the subject and a bioelectrical impedance that varies from hour to hour.

FIG. 22 is a flowchart showing the operation procedures of the body fat measurement device in measuring the visceral fat area, the subcutaneous fat area, and the body fat percentage using the body fat measurement device according to the third embodiment of the present invention.

FIG. 23 is a schematic perspective view showing an outer appearance structure of a body fat measurement device according to a fourth embodiment of the present invention.

FIG. 24A is a top view of the abdomen rear surface side base portion of the bioelectrical impedance measurement abdomen attachment unit shown in FIG. 23.

FIG. 24B is a side view of the abdomen rear surface side base portion of the bioelectrical impedance measurement abdomen attachment unit shown in FIG. 23.

FIG. 25 is a cross-sectional view of the abdomen rear surface side base portion of the bioelectrical impedance measurement abdomen attachment unit shown in FIG. 23.

FIG. 26A is a top view of the abdomen front surface side base portion of the bioelectrical impedance measurement abdomen attachment unit shown in FIG. 23.

FIG. 26B is a side view of a portion excluding the electrode film of the abdomen front surface side base portion of the bioelectrical impedance measurement abdomen attachment unit shown in FIG. 23.

FIG. 27 is a perspective view of a wind-up mechanism shown in FIG. 25.

FIG. 28 is a schematic cross-sectional view showing a state where the bioelectrical impedance measurement abdomen attachment unit according to the fourth embodiment of the present invention is attached to the abdomen of the subject.

FIG. 29 is a schematic cross-sectional view showing a state in which the bioelectrical impedance measurement abdomen attachment unit according to a variant of the fourth embodiment of the present invention is attached to the subject.

### DESCRIPTION OF THE REFERENCE NUMERALS

1A, 1B, 1C, 1D body fat measurement device
10 control unit
11 calculation processing section
12 impedance measuring portion
13 body fat mass calculating portion
14 total fat mass calculating part
15 site type fat mass calculating part
16 visceral fat mass calculating part
17 subcutaneous fat mass calculating part
18 breathing state detecting portion
21 constant current generation unit
22 terminal switching unit
23 potential difference detection unit
24 physical information measurement unit
25 subject information input unit
26 display unit
27 operation unit
28 power supply unit
29 memory
30 body peripheral length measurement unit
100A, 100A1, 100A2, 100B, 100C, 100D, 100D1 impedance measurement abdomen attachment unit
101 device main body
110A, 110A1, 110D, 110D1 abdomen rear surface side base portion
112 support base
112a one end
112b other end
112c, 112d slit
113 projection
114 upper surface
116a, 116b guide unit
117 grip portion
120A, 120A1, 120A2, 120D abdomen front surface side base portion
122 sheet
122a one end
122b other end
123 positioning through-hole
126 electrode film
127 positioning through-hole
128, 129 electrode
130, 130a, 130b wind-up unit
131, 131a, 131b grip portion
133, 133a, 133b chassis
134a, 134b support shaft
135a1, 135b1 shaft
135a, 135b reel
136a, 136b photoelectric sensor
137a1, 137a2, 137a3, 137b1, 137b2, 137b3, 137b4, 137b5 roller
138a, 138b biasing portion
139a, 139b rotary encoder
141a, 141b abdomen rear surface side belt member
142a1, 142b1 lock projection
142a, 142b connection tool
143a, 143b abdomen front surface side belt member
143a1, 143b1 encoder strip
144a, 144b connection tool
144a1, 144b1 lock recess
145a, 145b belt member
146a, 146b connection tool
146a1, 146b1 lock recess
147a, 147b lock projection
151 body peripheral length measurement circuit
200 attachment tool
300 abdomen
301 umbilicus
A11, A12, A21, A22 abdominal electrode
F11, F12, F21, F22 lower limb electrode
H11, H12, H21, H22 upper limb electrode

### BEST MODE FOR CARRYING OUT THE INVENTION

Embodiments of the present invention will be described in detail below with reference to the drawings. In each embodiment shown below, a bioelectrical impedance measurement body attachment unit aimed to be attached to an abdomen of the body of the subject and a body fat measurement device equipped with the same are illustratively described as the bioelectrical impedance measurement body attachment unit applied with the present invention and the body fat measurement device. The body fat measurement device in each embodiment described below is configured to be able to individually measure a visceral fat mass and a subcutaneous fat mass in particular, but may be configured not only to measure the visceral fat mass and the subcutaneous fat mass but also to measure a fat mass (total fat mass) of the entire body and a fat mass (fat mass of upper limb and lower limb, fat mass of body, etc.) by specific sites of the body.

First, prior to describing the bioelectrical impedance measurement abdomen attachment unit and the body fat measurement device quipped with the same in each embodiment of the present invention, various terms representing a site of the body will be defined. “Body” refers to a portion excluding a head, a neck, and four limbs of the body, and a portion corresponding to a so-called trunk of the body including a chest and the abdomen. “Body” includes a body front surface and a body rear surface as surfaces thereof, where the “body front surface” is the body surface of the portion visible when the subject is observed from the front side of the surface of the body of the subject, whereas the “body rear surface” is the body surface of the portion visible when the subject is observed from the back surface side. “Abdomen” is the portion positioned on the lower limb side when the body is divided to the portion positioned on the neck side (i.e., chest), and the portion positioned on the lower limb side, and surfaces thereof include an abdomen front surface and an abdomen rear surface for the surface. The “abdomen front surface” is the body surface of the portion visible when the subject is observed from the front surface side of the surface of the abdomen of the subject, and the “abdomen rear surface” is the body surface of the portion visible when the subject is observed from the rear surface side of the surface of the abdomen of the subject. The “site distant from the abdomen” includes the upper limb including an upper arm, a forearm, a wrist, and fingers, the chest distant by greater than or equal to a predetermined distance (e.g., about 10 cm) from a portion where a diaphragm is positioned, the neck and the head, and the lower limb including a thigh, a lower thigh, an ankle, and toes. “Body axis” refers to an axis extending in a direction substantially perpendicular to a transverse section of the abdomen of the subject.

### First Embodiment

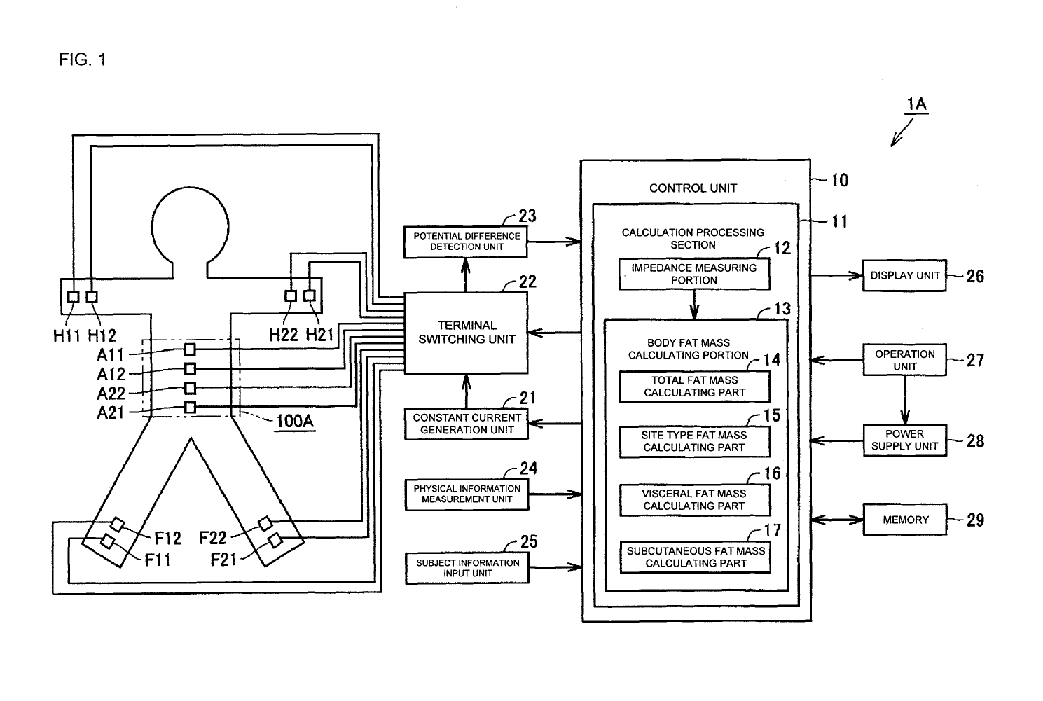

FIG. 1 is a view showing function blocks of a body fat measurement device according to a first embodiment of the present invention. First, a configuration of the function blocks of a body fat measurement device 1A according to the present embodiment will be described with reference to FIG. 1.

As shown in FIG. 1, a body fat measurement device 1A according to the present embodiment mainly includes a control unit 10, a constant current generation unit 21, a terminal switching unit 22, a potential difference detection unit 23, a physical information measurement unit 24, a subject information input unit 25, a display unit 26, an operation unit 27, a power supply unit 28, a memory 29, and a plurality of impedance measurement electrodes A11, A12, A21, A22, H11, H12, H21, H22, F11, F12, F21, and F22 attached to the body. The control unit 10 includes a calculation processing section 11. The calculation processing section 11 includes an impedance measuring portion 12, and a body fat mass calculating portion 13.

The control unit 10 is configured by a CPU (Central Processor Unit) and the like, and controls the overall body fat measurement device 1A. Specifically, the control unit 10 sends a command to the various types of function blocks described above or performs various types of calculation processes based on the obtained information. The various types of calculation processes are performed by the calculation processing section 11 arranged in the control unit 10.

The plurality of impedance measurement electrodes includes abdominal electrodes A11, A12, A21, A22 to be attached to the abdomen of the subject, upper limb electrodes H11, H12, H21, H22 to be attached to the upper limb of the subject, and lower limb electrodes F11, F12, F21, F22 to be attached to the lower limb of the subject.

The abdominal electrodes A11, A12, A21, A22 are arranged in the bioelectrical impedance measurement abdomen attachment unit 100A wrapped around and attached to the abdomen of the subject, where each electrode is attached to the surface of the abdomen of the subject while being aligned along the body axis direction by attaching the bioelectrical impedance measurement abdomen attachment unit 100A to the abdomen of the subject. The abdominal electrodes A11, A12, A21, A22 may be attached to the abdomen front surface of the subject, or may be attached to the abdomen rear surface of the subject. An abdominal electrode group, where four abdominal electrodes A11, A12, A21, A22 form one set, may be attached to the abdomen in plural sets. In such a case, the abdominal electrode group of all of the sets may be attached to only one of either the abdomen front surface or the abdomen rear surface, or the abdominal electrode group of some sets may be attached to the abdomen front surface and the abdominal electrode group of the remaining sets may be attached to the abdomen rear surface.

The upper limb electrodes H11, H12, H21, H22 are attached to one of the sites of the upper limb corresponding to the site distant from the abdomen of the subject, and one pair thereof is suitably attached to the surface of the wrist of the right hand and the surface of the wrist of the left hand, respectively. The lower limb electrodes F11, F12, F21, F22 are attached to one of the sites of the lower limb corresponding to the site distant from the abdomen of the subject, and one pair thereof is suitably attached to the surface of the ankle of the right foot and the surface of the ankle of the left foot, respectively. The abdominal electrodes A11, A12, A21, A22, the upper limb electrodes H11, H12, H21, H22, and the lower limb electrodes F11, F12, F21, F22 are respectively electrically connected to the terminal switching unit 22.

The terminal switching unit 22 is configured by a relay circuit, and the like, and electrically connects a specific electrode selected from the plurality of impedance measurement electrodes and the constant current generation unit 21, and electrically connects a specific electrode selected from the plurality of impedance measurement electrodes and the potential difference detection unit 23 based on a command inputted from the control unit 10. Thus, the electrode electrically connected to the constant current generation unit by the terminal switching unit 22 functions as a constant current application electrode, and the electrode electrically connected to the potential difference detection unit 23 by the terminal switching unit 22 functions as a potential difference detection electrode. The electrical connection by the terminal switching unit 22 is switched in various manners during the measurement operation. Normally, the constant current application electrode and the potential difference detection electrode are respectively configured by a pair of electrodes, where each of the pair of electrodes as referred to herein includes both single electrode or a plurality of electrodes. In other words, each of the pair of electrodes can be configured by handling even the separately and independently arranged electrode in an electrically equivalent manner.

The constant current generation unit 21 generates a constant current based on a command inputted from the control unit 10, and supplies the generated constant current to the constant current application electrode through the terminal switching unit 22. A high frequency current (e.g., 50 kHz, 500 pA) suitably used to measure the body composition information is selected for the constant current generated in the constant current generation unit 21. Thus, the constant current is applied to the subject through the constant current application electrode.

The potential difference detection unit 23 detects a potential difference between the electrodes (i.e., potential difference detection electrode) electrically connected to the potential difference detection unit 23 by the terminal switching unit 22, and outputs the detected potential difference to the control unit 10. In this manner, the potential difference between the potential difference detection electrodes with the constant current applied to the subject is detected.

The physical information measurement unit 24 and the subject information input unit 25 are sites for obtaining subject information used in the calculation process performed in the body fat mass calculating portion 13 of the calculation processing section 11. The “subject information” refers to information related to the subject, and includes at least one of the information of age, sex, or physical information. The “physical information” includes information related to a size at a specific site of the body of the subject (e.g., information including at least one of body peripheral length (waist length) and abdomen lateral width, abdomen thickness, height, etc.), or information such as a weight. The physical information measurement unit 24 is a unit for automatically measuring the physical information of the subject, and outputs the detected physical information to the control unit 10. The subject information input unit 25 is a unit for inputting the subject information, and outputs the inputted subject information to the control unit 10.

In the function block diagram shown in FIG. 1, a case where both the physical information measurement unit 24 and the subject information input unit 25 are arranged in the body fat measurement device 1A has been described, but both the physical information measurement unit 24 and the subject information input unit 25 are not essential configurations. Whether or not to arrange the physical information measurement unit 24 and/or subject information input unit 25 is appropriately selected based on the type of subject information used in a calculation process performed in the calculation processing section 11 of the control unit 10. The physical information of the subject information may be automatically measured using the physical information measurement unit 24, and the measurement data may be used, or the subject himself/herself may input the information at the subject information input unit 25 without arranging the physical information measurement unit 24 and the input data may be used.

The calculation processing section 11 includes the impedance measuring portion 12 and the body fat mass calculating portion 13, as described above. The impedance measuring portion 12 calculates various types of bioelectrical impedances based on the current value of the constant current generated by the constant current generation unit 21, and the potential difference information inputted to the control unit 10 detected in the potential difference detection unit 23. The body fat mass calculating portion 13 calculates the body fat mass based on the bioelectrical impedance obtained by the impedance measuring portion 12 and the subject information inputted from the physical information measurement unit 24 and/or the subject information input unit 25. The body fat mass calculating portion 13 includes, for example, at least one of a total fat mass calculating part 14 for calculating the body fat mass of the entire body of the subject, a site type fat mass calculating part 15 for calculating the fat mass by specific site of the body of the subject, a visceral fat mass calculating part 16 for calculating the visceral fat mass of the subject, and a subcutaneous fat mass calculating part 17 for calculating the subcutaneous fat mass at the abdomen of the subject.

The display unit 26 displays information of various types of body fat mass calculated by the body fat mass calculating portion 13. An LCD (Liquid Crystal Display), and the like can be used for the display unit 26. The fat mass displayed on the display unit 26 may be total fat mass, i.e., the fat mass of the entire body of the subject, the site type fat mass, i.e., the fat mass of specific site of the body of the subject, the visceral fat mass, the subcutaneous fat mass at the abdomen, and the like. The “fat mass” refers to an index indicating the fat mass represented by a weight of fat, area of fat, volume of fat, fat level, and the like. In particular, the “visceral fat mass” refers to an index represented by at least one of a weight of visceral fat, area of visceral fat, volume of visceral fat, and visceral fat level; and the “subcutaneous fat mass” refers to an index represented by at least one of a weight of subcutaneous fat, area of subcutaneous fat, volume of subcutaneous fat, and subcutaneous fat level.

The operation unit 27 is a unit for the subject to input a command to the body fat measurement device 1A, and is configured by a key and the like that can be pushed by the subject.

The power supply unit 28 is a unit for supplying power to the control unit 10, and includes an internal power supply such as a battery and an external power supply such as a commercial power supply.

The memory 29 is a unit for storing various types of data and program related to the body fat measurement device 1A, and stores the subject information, the various types of calculated body fat mass, body fat measurement program for executing the body fat measurement process described below, and the like.

One example of the calculation process performed in the body fat measurement device 1A according to the present embodiment will now be described. As described above, various types of body fat mass can be measured by the body fat mass calculating portion 13 in the body fat measurement device 1A according to the present embodiment, where particularly described below by way of example is the calculation process performed when calculating the area of the visceral fat serving as an index indicating the visceral fat mass, the area of the subcutaneous fat serving as an index indicating the subcutaneous fat mass, and the body fat percentage serving as an index indicating the relationship of the body fat mass and the weight.

With reference to FIG. 1, the impedance measuring portion 12 calculates two types of bioelectrical impedances based on the current value of the constant current generated by the constant current generation unit 21 and the potential difference detected by the potential difference detection unit 23. One of the two types of bioelectrical impedances is a bioelectrical impedance Zt reflecting the fat free mass at the abdomen of the subject. The other bioelectrical impedance is a bioelectrical impedance Zs reflecting the subcutaneous fat mass at the abdomen of the subject.

The visceral fat mass calculating part 16 calculates a visceral fat area Sv (unit: cm²) of the subject based on the calculated two types of impedances Zt, Zs, and a body peripheral length W, which is one of the physical information of the subject. Specifically, the visceral fat area Sv is calculated by the following equation (1) expressing the relationship of the two types of impedances Zt, Zs and the body peripheral length W of the subject, and the visceral fat area Sv.

Sv=a×W²−b×(1/Zt)−c×W×Zs−d (1)

(where, a, b, c, d: coefficient)

The subcutaneous fat mass calculating part 17 calculates a subcutaneous fat area Ss (unit: cm²) of the subject based on the calculated bioelectrical impedance Zs and the body peripheral length W, which is one of the physical information of the subject. Specifically, the subcutaneous fat area Ss is calculated by the following equation (2) expressing the relationship of the bioelectrical impedance Zs and the body peripheral length W of the subject, and the subcutaneous fat area Ss.

Ss=e×W×Zs+f (2)

(where, e, f: coefficients)

Although the calculation of the visceral fat area Sv and the calculation of the subcutaneous fat area Ss are not directly related, the total fat mass calculating part 14 calculates a fat free mass FFM (unit: kg) based on the calculated bioelectrical impedance Zt and a height H, which is one of the physical information of the subject when calculating the fat free mass. Specifically, the fat free mass FFM is calculated by the following equation (3) expressing the relationship of the bioelectrical impedance Zt and the height H of the subject, and the fat free mass FFM.

FFM=i×H²/Zt+j (3)

(where i, j: coefficients)

The coefficients in each equation (1), (2), and (3) as above are defined by a regression equation based on the measurement result of an MRI. The coefficients in each equation (1), (2), and (3) may be defined by age and/or sex.

Furthermore, the total fat mass calculating part 14 calculates the body fat mass of the subject such as the body fat percentage (%) based on the calculated fat free mass FFM and the weight Wt, which is physical information, when calculating the body fat mass of the entire body of the subject. Specifically, for example, the body fat percentage is calculated by the following equation (4) based on the fat free mass FFM and the weight Wt of the subject.

Body fat percentage=(Wt−FFM)/Wt×100 (4)

Although the specific description will not be given, the body fat mass by sites of the body can be calculated based on the bioelectrical impedance, obtained by variously switching the current application electrode and the potential difference detection electrode, and the physical information of the subject.

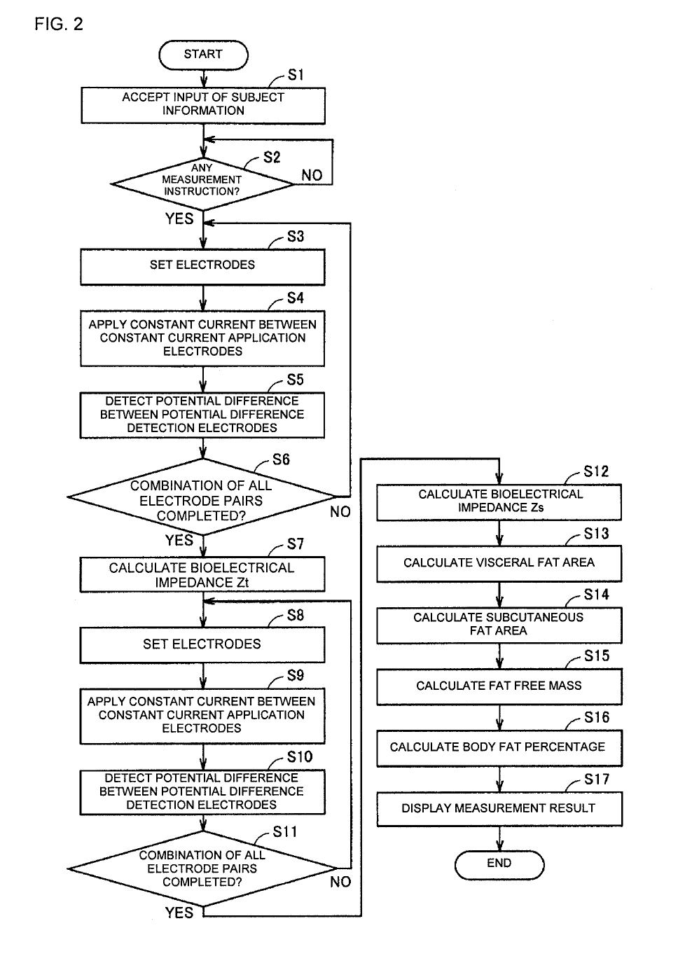

FIG. 2 is a flowchart showing the operation procedures of the body fat measurement device in measuring the visceral fat area, the subcutaneous fat area, and the body fat percentage using the body fat measurement device according to the present embodiment. The operation of the body fat measurement device 1A in measuring the visceral fat area, the subcutaneous fat area, and the body fat percentage using the body fat measurement device 1A will be described with reference to FIG. 2.

The process shown in the flowchart of FIG. 2 is stored in the memory 29 as a program in advance, where the control unit 10 including the calculation processing section 11 reads out and executes the program to realize the functions of the visceral fat area measurement process, the subcutaneous fat area measurement process, and the body fat percentage measurement process. The operation procedures described below are the operation procedures in the case where four sets of abdominal electrode groups, each set including four abdominal electrodes A11, A12, A21, A22 shown in the figure, are arranged parallel to each other in the body fat measurement device shown in FIG. 1.

With reference to FIG. 2, the control unit 10 accepts the input of the subject information containing the body peripheral length W, the height H, the weight Wt and the like serving as physical information (step S1). The accepted subject information is temporarily saved in the memory 29, for example. If a configuration of automatically measuring specific physical information of the subject information using the physical information measurement unit 24 is adopted, the physical information measured by the physical information measurement unit 24 is inputted to the control unit 10.

The control unit 10 determines whether or not an instruction to start the measurement is made (step S2). The control unit 10 waits until the instruction to start the measurement is made (NO in step S2). The control unit 10 sets the electrode (step S3) when detecting the instruction to start the measurement (YES in step S2).

In step S3, the control unit 10 selects, for example, a pair of upper limb electrode H11 and lower limb electrode F11 and a pair of upper limb electrode H21 and lower limb electrode F21 as the current application electrode pairs, and selects one pair of abdominal electrodes A11, A21 in one abdominal electrode group of the four sets of abdominal electrode groups as the potential difference detection electrode pair. The terminal switching unit 22 electrically connects the pair of upper limb electrode H11 and lower limb electrode E11 and the pair of upper limb electrode H21 and lower limb electrode F21 with the constant current generation unit 21, and electrically connects the pair of abdominal electrodes A11, A21 with the potential difference detection unit 23 based on the control of the control unit 10. In this case, the terminal switching unit 22 cuts the electrical connection of the non-selected electrode and the constant current generation unit 21 and the potential difference detection unit 23 based on the control of the control unit 10.

The constant current generation unit 21 flows a constant current between the upper limb and the lower limb based on the control of the control unit 10. For instance, the constant current generation unit 21 flows a constant current from the upper limb electrode H11 and the upper limb electrode H21 to the lower limb electrode F11 and the lower limb electrode F21 (step S4). In this case, the terminal switching unit 22 preferably has a configuration of short circuiting the upper limb electrode H11 and the upper limb electrode H21 and short circuiting the lower limb electrode F11 and the lower limb electrode F21. The constant current generation unit 21 and the terminal switching unit 22 may have a configuration of flowing a constant current from either one of the upper limb electrodes H11, H21 to either one of the lower limb electrodes F11, F21.

In this state, the potential difference detection unit 23 detects the potential difference between the abdominal electrodes A11, A21 based on the control of the control unit 10 (step S5).

The control unit 10 determines whether or not the detection of the potential difference is completed for the combinations of all electrode pairs defined in advance (step S6). The control unit 10 proceeds to step S3 when determined that the detection of the potential difference is not completed for the combination of all of the electrode pairs defined in advance (NO in step S6). The control unit 10 proceeds to step S7, to be hereinafter described, when determined that the detection of the potential difference is completed for the combination of all of the electrode pairs defined in advance (YES in step S6).

In this manner, the control unit 10 selects the abdominal electrodes A11, A21 of another abdominal electrode group in order as the potential difference detection electrode pair. That is, the terminal switching unit 22 electrically connects the abdominal electrodes A11, A21 of another abdominal electrode group with the potential difference detection unit 23 in order based on the control of the control unit 10 (step S3). The potential difference detection unit 23 then detects the potential difference between the abdominal electrodes A11, A21 of another abdominal electrode group in order based on the control of the control unit 10 (step S5).

When the detection of the potential difference is completed for the combination of the abdominal electrodes A11, A21 in all of the abdominal electrode groups (YES in step S6), the impedance measuring portion 12 calculates the bioelectrical impedances Zt1 to Zt4 based on the current value of the constant current generated by the constant current generation unit 21 and flowed through the body and each potential difference detected by the potential difference detection unit 23 (step S7). The values of the bioelectrical impedances Zt1 to Zt4 calculated by the impedance measuring portion 12 are temporarily saved in the memory 29, for example.

The control unit 10 then sets the electrodes again (step S8). More specifically, the control unit 10 selects the pair of abdominal electrodes A11, A21 in one abdominal electrode group of the four sets of abdominal electrode groups as the current application electrode pair, and selects the pair of abdominal electrodes A12, A22 in the abdominal electrode group as the potential difference detection electrode pair. The terminal switching unit 22 electrically connects the pair of abdominal electrodes A11, A21 with the constant current generation unit 21 and electrically connects the pair of abdominal electrodes A12, A22 with the potential difference detection unit 23 based on the control of the control unit 10. In this case, the terminal switching unit 22 cuts the electrical connection of the non-selected abdominal electrode, the upper limb electrode and the lower limb electrode and the constant current generation unit 21 and the potential difference detection unit 23 based on the control of the control unit 10.

The constant current generation unit 21 flows a constant current between the abdominal electrodes A12, A22 based on the control of the control unit 10 (step S9).

In this state, the potential difference detection unit 23 detects the potential difference between the abdominal electrodes A21, A21 based on the control of the control unit 10 (step S10).

The control unit 10 then determines whether or not the detection of the potential difference is completed for the combinations of all electrode pairs defined in advance (step S11). The control unit 10 proceeds to step S8 when determined that the detection of the potential difference is not completed for the combination of all of the electrode pairs defined in advance (NO in step S11). The control unit 10 proceeds to step S12, to be hereinafter described, when determined that the detection of the potential difference is completed for the combination of all of the electrode pairs defined in advance (YES in step S11).

In this manner, the control unit 10 selects the abdominal electrodes A11, A21 of another abdominal electrode group as the current application electrode pair, and selects the abdominal electrodes A12, A22 in the relevant abdominal electrode group in order as the potential difference detection electrode pair. In other words, the terminal switching unit 22 electrically connects the abdominal electrodes A11, A21 of another abdominal electrode group with the constant current generation unit 21 in order, and electrically connects the abdominal electrodes A12, A22 in the relevant abdominal electrode group with the potential difference detection unit 23 in order based on the control of the control unit 10 (step S8). The potential difference detection unit 23 then flows a constant current between the abdominal electrodes A11, A12 in another abdominal electrode group (step S9), and detects the potential difference between the abdominal electrodes A12, A22 in the relevant abdominal electrode group in order based on the control of the control unit 10 (step S10).

When the application of the current and the detection of the potential difference for the combination of the electrode pairs in all of the abdominal electrode groups are completed (YES in step S11), the impedance measuring portion 12 calculates bioelectrical impedances Zs1 to Zs4 (step S12) based on the current value of the constant current generated by the constant current generation unit 21 and flowed through the body and each potential difference detected by the potential difference detection unit 23. The values of the bioelectrical impedances Zs1 to Zs4 calculated by the impedance measuring portion 12 are temporarily saved in the memory 29, for example.

The visceral fat mass calculating part 16 then calculates the visceral fat area Sv based on the body peripheral length W of the physical information accepted by the control unit 10 in step S1, the calculated bioelectrical impedances Zt1 to Zt4, and the bioelectrical impedances Zs1 to Zs4 (step S13). The visceral fat area Sv is calculated by equation (1). In the case where four sets of abdominal electrode groups, each set including four abdominal electrodes A11, A12, A21, A22, are arranged parallel to each other, the average value of the four bioelectrical impedances Zt1 to Zt4 and the average value of the four bioelectrical impedances Zs1 to Zs4, for example, are respectively substituted to equation (1).

The subcutaneous fat mass calculating part 17 then calculates the subcutaneous fat area Ss based on the body peripheral length W of the physical information accepted by the control unit 10 in step S1, and the calculated bioelectrical impedances Zs1 to Zs4 (step S14). The subcutaneous fat area Ss is calculated by equation (2). In the case where four sets of abdominal electrode groups, each set including four abdominal electrodes A11, A12, A21, A22, are arranged parallel to each other, the average value of the four bioelectrical impedances Zs1 to Zs4 is substituted to the bioelectrical impedance Zs of equation (2).

The total fat mass calculating part 14 calculates the fat free mass FFM based on the height H of the physical information accepted by the control unit 10 in step S1 and the calculated bioelectrical impedance Zt (step S15). The fat free mass FFM is calculated by equation (3).

The total fat mass calculating part 14 calculates the body fat percentage based on the weight Wt of the physical information accepted by the control unit 10 in step S1, and the fat free mass FFM calculated by the total fat mass calculating part 14 in step S15 (step S16). The body fat percentage is calculated from equation (4).

The display unit 26 displays each measurement result based on the control of the control unit 10 (step S17).

The body fat measurement device 1A then ends the body fat mass measurement process including the visceral fat area measurement process, the subcutaneous fat area measurement process, and the body fat percentage measurement process. A typical value of the bioelectrical impedances Zt1 to Zt4 is about 5Ω. A typical value of the bioelectrical impedances Zs1 to Zs4 is about 800.

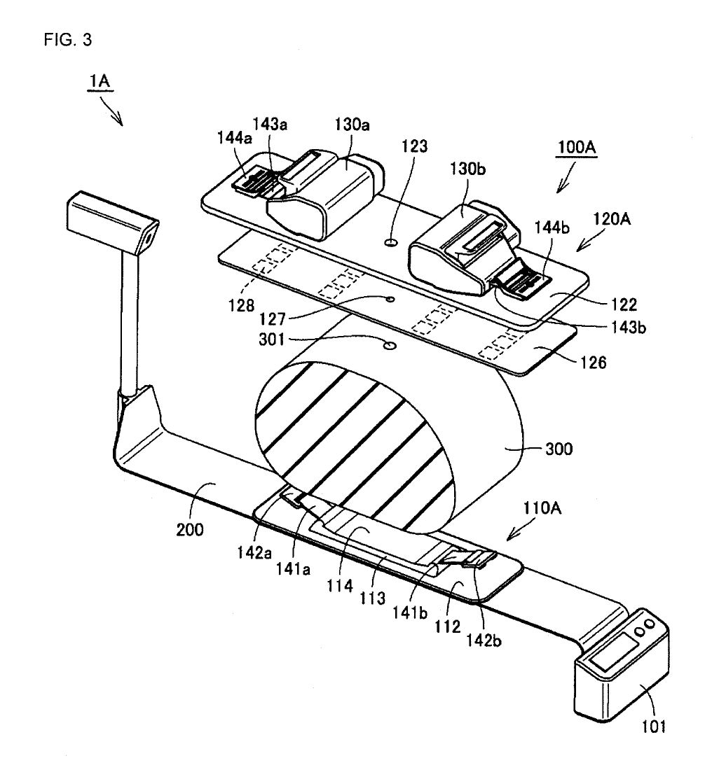

FIG. 3 is a schematic perspective view showing an outer appearance structure of the body fat measurement device according to the present embodiment. The outer appearance structure of the body fat measurement device 1A according to the present embodiment will be described with reference to FIG. 3. The body fat measurement device 1A described below is configured with four sets of abdominal electrode groups, each set including four illustrated abdominal electrodes A11, A12, A21, A22, arranged parallel to each other in the body fat measurement device shown in FIG. 1.

As shown in FIG. 3, the body fat measurement device 1A according to the present embodiment mainly includes a device main body 101, a bioelectrical impedance measurement abdomen attachment unit 100A, a pair of bioelectrical impedance measurement upper limb attachment units (not shown), and a pair of bioelectrical impedance measurement lower limb attachment units (not shown).

The device main body 101 has a box-shaped outer shape, and includes the control unit 10, the constant current generation unit 21, the terminal switching unit 22, the potential difference detection unit 23, the subject information input unit 25, the display unit 26, the operation unit 27, the memory 29, and the like. The device main body 101 is arranged at an end of an attachment tool 200.

The bioelectrical impedance measurement abdomen attachment unit 100A is aimed to be attached when the subject takes a laid position on a bed (not shown). The bioelectrical impedance measurement abdomen attachment unit 100A includes an abdomen front surface side base portion 120A serving as a first base portion and an abdomen rear surface side base portion 110A serving as a second base portion. The abdomen rear surface side base portion 110A is placed on the abdomen rear surface of an abdomen 300 of the subject in the attached state. The abdomen front surface side base portion 120A is placed on the abdomen front surface of the abdomen 300 of the subject in the attached state. More specifically, the abdomen rear surface side base portion 110A is attached to a bed with the attachment tool 200 in advance, and is placed on the abdomen rear surface of the subject in a state the subject is laid down in a face-up position on the bed surface. The abdomen front surface side base portion 120A is placed on the abdomen front surface of the subject by being mounted on the abdomen front surface of the subject in a state the subject is laid down in the face-up position on the bed surface. The abdomen front surface side base portion 120A includes a sheet 122 serving as a first sheet-like member and an electrode film 126 serving as a second sheet-like member. Thus, the electrode film 126 can be used as a disposable type biological electrode by being replaced every time it is used by dividing the abdomen front surface side base portion 120A to the sheet 122 and the electrode film 126, thereby excelling in hygienic aspects.

The pair of bioelectrical impedance measurement upper limb attachment units and the pair of bioelectrical impedance measurement lower limb attachment units are respectively configured with a clip-shaped member capable of clipping the upper limb or the lower limb of the subject. The pair of bioelectrical impedance measurement upper limb attachment units include upper limb electrodes (upper limb electrodes H11, H12, H21, H22 described above) that can be arranged in contact with the surface of the upper limb of the subject, and are suitably attached to the wrist of the subject. The pair of bioelectrical impedance measurement lower limb attachment units includes lower limb electrodes (lower limb electrodes F11, F12, F21, F22 described above) that can be arranged in contact with the surface of the lower limb of the subject, and are suitably attached to the ankle of the subject.

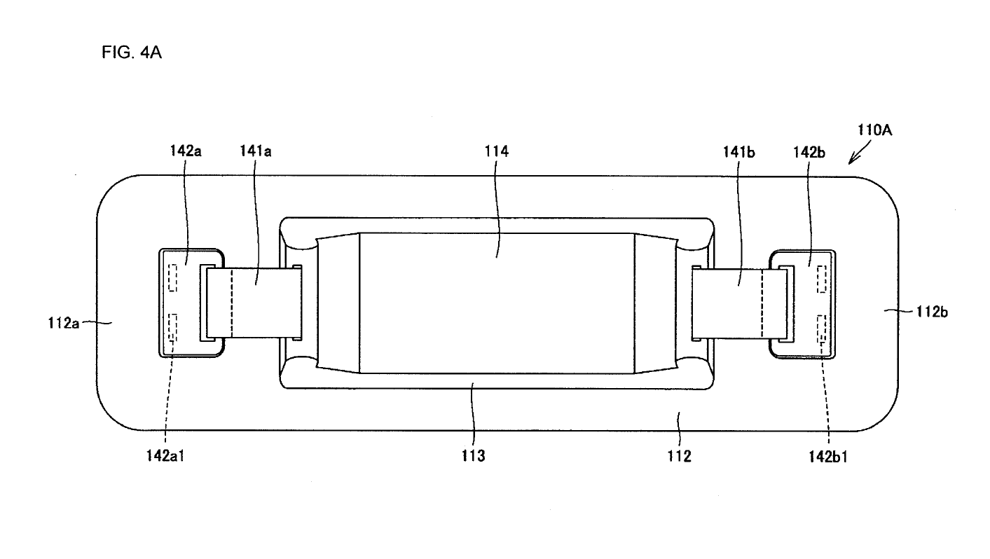

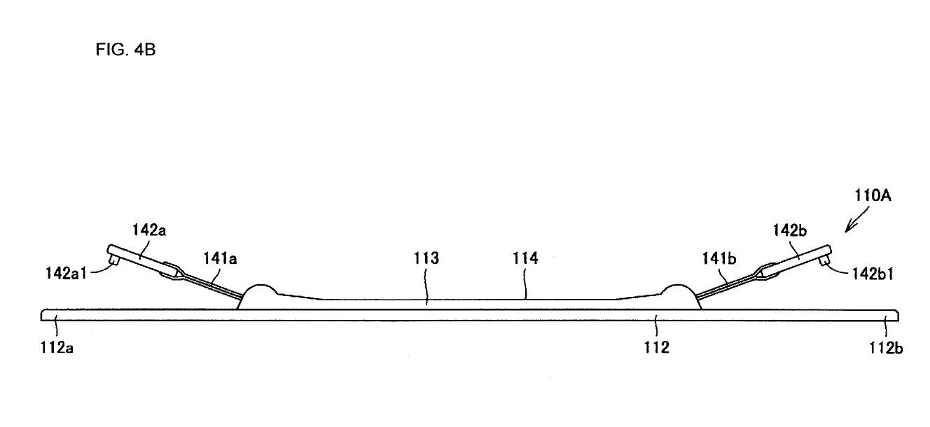

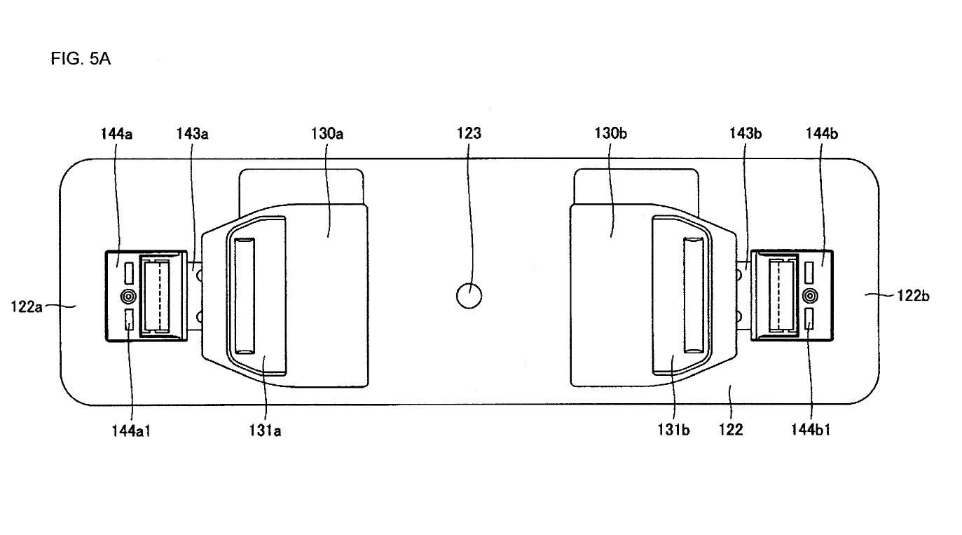

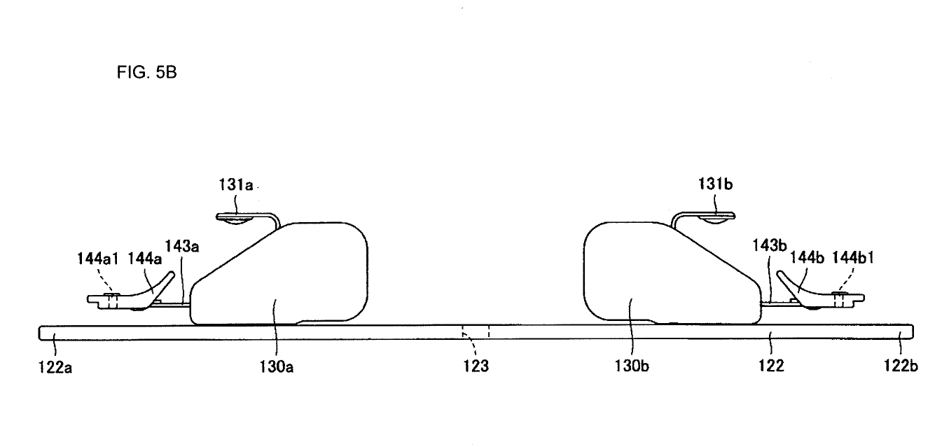

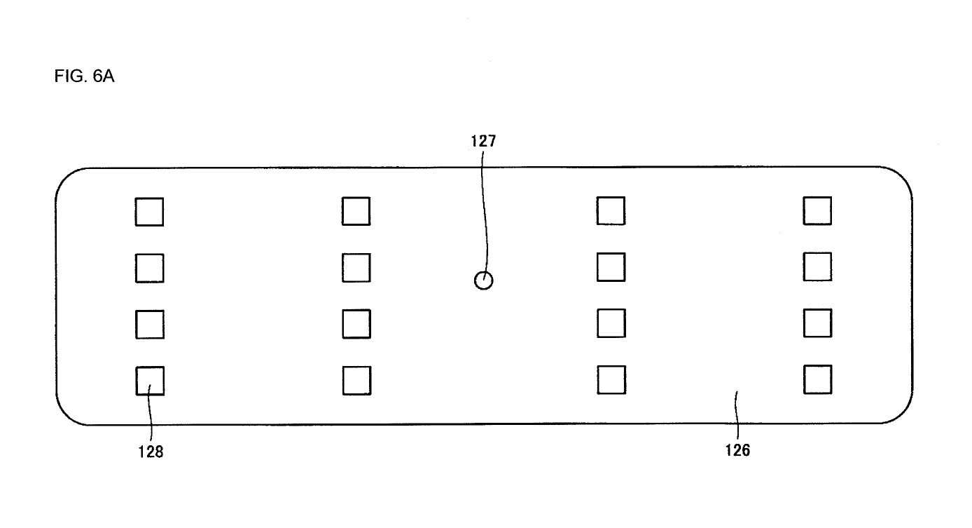

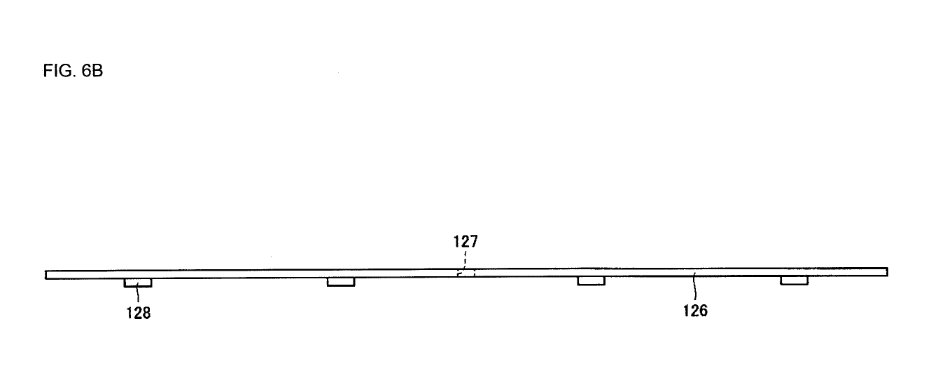

FIG. 4A is a top view of the abdomen rear surface side base portion of the bioelectrical impedance measurement abdomen attachment unit shown in FIG. 3, and FIG. 4B is a side view of the abdomen rear surface side base portion. FIG. 5A is a top view of the abdomen front surface side base portion of the bioelectrical impedance measurement abdomen attachment unit shown in FIG. 3, and FIG. 5B is a side view of the portion excluding the electrode film of the abdomen front surface side base portion. FIG. 6A is a bottom view of the electrode film of the abdomen front surface side base portion, and FIG. 6B is a side view of the electrode film. In the following, the outer appearance structure of the bioelectrical impedance measurement abdomen attachment unit 100A according to the present embodiment will be described in more detail with reference to FIG. 3.

As shown in FIGS. 3, 4A, and 4B, the abdomen rear surface side base portion 110A has a substantially rectangular outer shape having a direction corresponding to a waistline direction of the subject as the longitudinal direction in the attached state. The abdomen rear surface side base portion 110A mainly includes a plate-shaped support base 112, a projection 113 arranged projecting upward from the support base 112, and a pair of abdomen rear surface side belt members 141a, 141b arranged at the projection 113. The support base 112 has the lower surface fixed to the attachment tool 200, and includes one end 112a and the other end 112b in the longitudinal direction. The projection 113 is configured to receive the abdomen rear surface of the subject with the upper surface 114. The pair of abdomen rear surface side belt members 141a, 141b is arranged at both ends on the side facing the one end 112a and the other end 112b in the longitudinal direction of the support base 112 of the projection 113. Connection tools 142a, 142b are respectively attached to the end on the opposite side of the end of the side connected to the projection 113 of the pair of abdomen rear surface side belt members 141a, 141b. Lock projections 142a1, 142b1 are respectively arranged at predetermined positions of the connection tools 142a, 142b.

As shown in FIGS. 3, 5A, and 5B, the sheet 122 of the abdomen front surface side base portion 120A has a substantially rectangular outer shape having the direction corresponding to the waistline direction of the subject in the attached state as the longitudinal direction. The sheet 122 is made of a flexible material to deform along the shape of the abdomen front surface of the subject and fit to the abdomen front surface when placed on the abdomen front surface of the abdomen 300 of the subject. A positioning through-hole 123 serving as a mark for alignment with the umbilicus 301 of the subject in attachment is arranged substantially at the central part of the sheet 122.

A pair of wind-up units 130a, 130b is arranged at the upper surface of the sheet 122. The pair of wind-up units 130a, 130b are arranged lined in the longitudinal direction of the sheet 122, where one wind-up unit 130a is arranged at a portion closer to one end 122a in the longitudinal direction of the sheet 122 and the other wind-up unit 130b is arranged at a portion closer to the other end 122b in the longitudinal direction of the sheet 122. Each wind-up unit 130a, 130b includes a grip portion 131a, 131b at the upper surface, and interiorly includes a wind-up mechanism serving as a wind-up mechanism to be hereinafter described.

The side surface of the portion facing the one end 122a and the other end 122b in the longitudinal direction of the sheet 122 of the wind-up units 130a, 130b respectively includes an opening, so that the abdomen front surface side belt members 143a, 143b serving as the first belt member and the second belt member are respectively pulled out from the opening. Each abdomen front surface side belt member 143a, 143b has one end inserted into the wind-up unit 130a, 130b and fixed to the wind-up mechanism, to be hereinafter described, and the other end pulled out to the outside of the wind-up unit 130a, 130b. The connection tools 144a, 144b are attached to the other ends of the abdomen front surface side belt members 143a, 143b. Lock recesses 144a1, 144b1 capable of engaging with the lock projections 142a1, 142b1 of the connection tools 142a, 142b attached to the abdomen rear surface side belt members 141a, 141b are arranged at predetermined positions of the connection tools 144a, 144b.

As shown in FIGS. 3, 6A, and 6B, the electrode film 126 of the abdomen front surface side base portion has a substantially rectangular outer shape having the direction corresponding to the waist line direction of the subject in the attached state as the longitudinal direction. The electrode film 126 is made of a flexible material that deforms along the shape of the abdomen front surface of the subject and fits to the abdomen front surface when placed on the abdomen front surface of the abdomen 300 of the subject. A positioning through-hole 127 serving as a mark for alignment with the umbilicus 301 of the subject in attachment is arranged substantially at the central part of the electrode film 126. A plurality of electrodes 128 is arranged in a projecting manner at the lower surface of the electrode film 126. The plurality of electrodes 128 correspond to the abdominal electrodes A11, A12, A21, A22, and are arranged in contact with the abdomen front surface of the subject in the attached state.

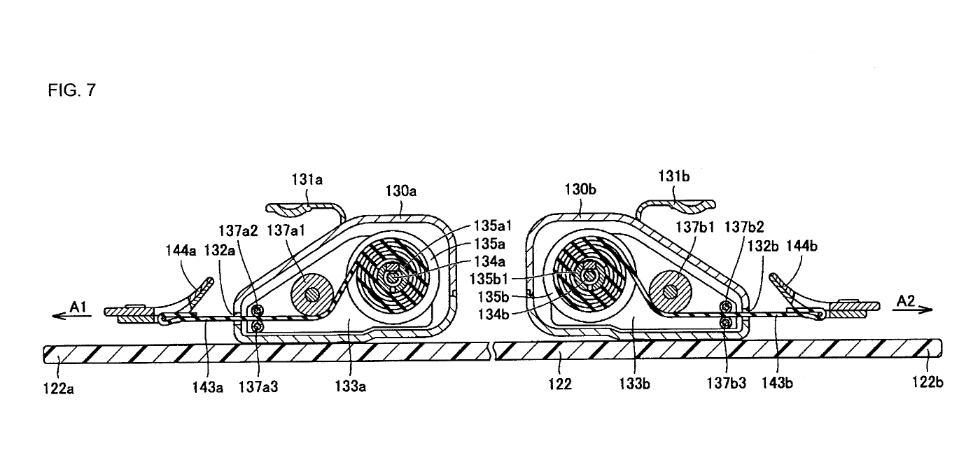

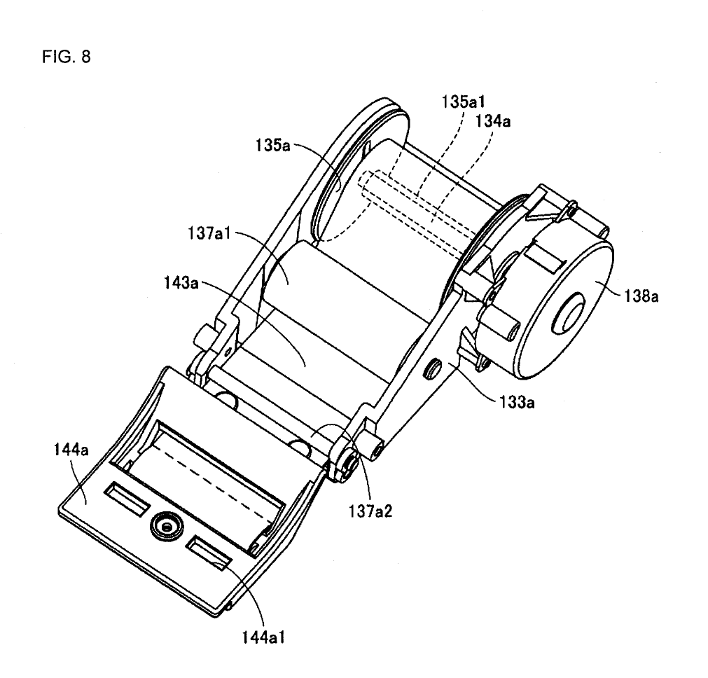

FIGS. 7 and 8 are views describing the structure of the wind-up mechanism arranged inside the wind-up unit, where FIG. 7 is a cross-sectional view of the portion excluding the electrode film of the abdomen front surface side base portion, and FIG. 8 is a perspective view of the wind-up mechanism. In FIG. 8, only the wind-up unit 130a arranged in the wind-up unit 130a of the wind-up mechanism arranged in the wind-up units 130a, 130b is illustrated for the sake of simplifying the description. The structure of the wind-up mechanism arranged in the abdomen front surface side base portion 120A will now be described with reference to such figures.

As shown in FIGS. 7 and 8, the chassis 133a is arranged inside the wind-up unit 130a, and the wind-up mechanism is assembled to the chassis 133a. The wind-up mechanism is configured by a support shaft 134a, a reel 135a, a biasing portion 138a and rollers 137a1, 137a2, and 137a3. The support shaft 134a is axially supported by the chassis 133a, and a shaft 135a1 of the reel 135a is externally inserted to the support shaft 134a. The reel 135a is fixed with one end of the abdomen front surface side belt member 143a, and the abdomen front surface side belt member 143a is wrapped around thereto. The biasing portion 138a interiorly accommodates a protrusion spring (not shown) serving as a first biasing portion, which protrusion spring is connected to the reel 135a. The rollers 137a1, 137a2, and 137a3 are supported by the chassis 133a, and comes into contact with the main surface of the abdomen front surface side belt member 143a of the portion pulled out from the reel 135a to guide the abdomen front surface side belt member 143a.

According to the wind-up mechanism of the above configuration, the abdomen front surface side belt member 143a is configured to be able to be pulled out from the reel 135a towards a direction of arrow A1 in FIG. 7, and the abdomen front surface side belt member 143a is wound up by the reel 135a by the elastic force of the protrusion spring serving as the first biasing portion when a force is not applied on the abdomen front surface side belt member 143a. As shown in FIG. 7, the wind-up mechanism similar to the wind-up mechanism arranged inside the wind-up unit 130a is also arranged inside the wind-up unit 130b. Therefore, the abdomen front surface side belt member 143b is configured to be able to be pulled out from the reel 135b towards a direction of arrow A2 in FIG. 7, and the abdomen front surface side belt member 143b is wound up by the reel 135b by the elastic force of the protrusion spring serving as the second biasing portion when a force is not applied on the abdomen front surface side belt member 143b. The directions A1, A2 in which the pair of abdomen front surface side belt members 143a, 143b are pulled out from the wind-up units 130a, 130b are opposite directions along the peripheral direction of the abdomen of the subject.

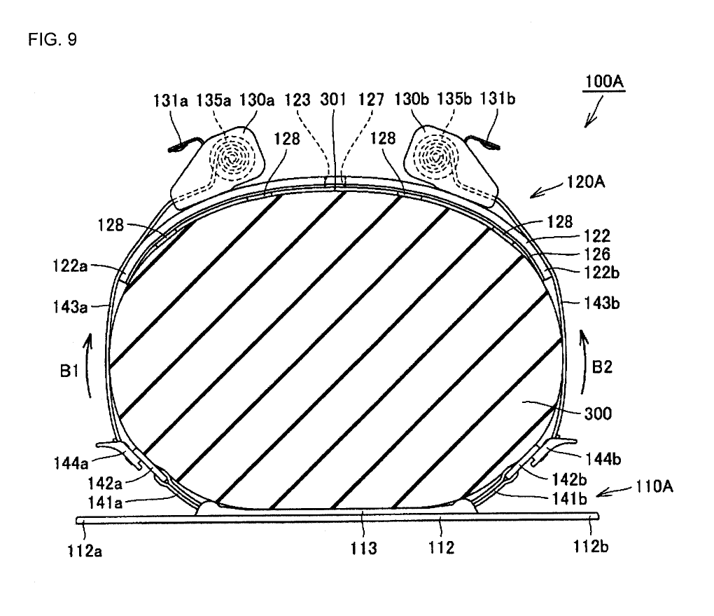

FIG. 9 is a schematic cross-sectional view showing a state in which the bioelectrical impedance measurement abdomen attachment unit of the above configuration is attached to the abdomen of the subject. In the following, the procedures for attaching the bioelectrical impedance measurement abdomen attachment unit 100A according to the present embodiment to the abdomen of the subject and the attached state will be described with reference to the figure.

When attaching the bioelectrical impedance measurement abdomen attachment unit 100A according to the present embodiment, the subject first lays face up on the bed. In this case, the abdomen 300 of the subject is positioned on the abdomen rear surface side base portion 110A arranged on the bed. The electrode film 126 is then mounted on the front surface of the abdomen of the subject. In this case, alignment of the positioning through-hole 127 formed in the electrode film 126 and the umbilicus 301 of the subject is carried out. The sheet 122 arranged with the wind-up units 130a, 130b is then mounted on the electrode film 126 placed on the front surface of the abdomen of the subject. In this case, alignment of the positioning through-hole 123 formed in the sheet 122 and the umbilicus of the subject is carried out. The abdomen front surface side base portion 120A is preferably mounted to the front surface of the abdomen of the subject by a helper, in which case, the task is very easily performed if the abdomen front surface side base portion 120A is mounted to the front surface of the abdomen of the subject of the sheet 122 using the grip portions 131a, 131b arranged at the wind-up units 130a, 130b.

Subsequently, the abdomen front surface side belt members 143a, 143b are respectively pulled out from the wind-up units 130a, 130b, and the connection tools 144a, 144b arranged at the distal ends are connected to the connection tools 142a, 142b arranged at the distal ends of the abdomen rear surface side belt members 141a, 141b. Such connections are carried out by locking the lock projections 142a1, 142b1 arranged at the connection tools 142a, 142b to the lock recesses 144a1, 144b1 arranged at the connection tools 144a, 144b. The attachment of the bioelectrical impedance measurement abdomen attachment unit 100A to the abdomen 300 of the subject is thereby completed.

As shown in FIG. 9, when the bioelectrical impedance measurement abdomen attachment unit 100A is attached to the abdomen 300 of the subject, the abdomen front surface side belt members 143a, 143b are respectively pulled towards directions of arrows B1, B2 shown in the figure by the biasing force of the protrusion spring arranged in the wind-up mechanism. In the attached state, the abdomen front surface side belt members 143a, 143b are respectively pulled out from the reels 135a, 135b against the biasing force of the protrusion spring serving as the first biasing portion and the second biasing portion with increase in the body peripheral length of the subject when the subject performs the inhaling motion. When the subject performs the exhaling motion, the abdomen front surface side belt members 143a, 143b are wound up by the reels 135a, 135b by the biasing force of the protrusion spring serving as the first biasing portion and the second biasing portion with decrease in the body peripheral length of the subject.

According to the bioelectrical impedance measurement abdomen attachment unit 100A and the body fat measurement device 1A equipped with the same according to the present embodiment described above, the abdomen front surface side belt members 143a, 143b are constantly pulled in the attached state. Therefore, the bioelectrical impedance measurement abdomen attachment unit 100A constantly fits to the abdomen 300 of the subject even when the subject performs the breathing motion. As the abdomen 300 of the subject is tightened at a substantially constant tightening strength by the bioelectrical impedance measurement abdomen attachment unit 100A based on the biasing force of the protrusion spring, the plurality of electrodes 128 arranged at the electrode film 126 can be pressed against the abdomen 300 of the subject at a substantially constant load. The tensile load applied on the bioelectrical impedance measurement abdomen attachment unit 100A when the pressing force of the electrode 128 with respect to the abdomen 300 of the subject is optimized is between about 1.0 kgf to 2.0 kgf, and preferably 1.5 kgf.

In the present embodiment, the bioelectrical impedance measurement abdomen attachment unit 100A attached to the abdomen 300 of the subject is divided into the abdomen rear surface side base portion 110A to be placed on the abdomen rear surface side of the subject and the abdomen front surface side base portion 120A to be placed on the abdomen front surface side of the subject, where the portion closer to one end of the abdomen rear surface side base portion 110A and the portion closer to one end of the abdomen front surface side base portion 120A are coupled by the abdomen front surface side belt member 143a and the protrusion spring serving as a first coupling mechanism, and the portion closer to the other end of the abdomen rear surface side base portion 110A and the portion closer to the other end of the abdomen front surface side base portion 120A are coupled by the abdomen front surface side belt member 143b and the protrusion spring serving as a second coupling mechanism, so that the bioelectrical impedance measurement abdomen attachment unit 100A can be very easily attached to the abdomen 300 of the subject. Therefore, a troublesome attachment task is unnecessary, and the bioelectrical impedance measurement abdomen attachment unit excelling in handleability and the body fat measurement device equipped with the same can be obtained.

Furthermore, according to the bioelectrical impedance measurement abdomen attachment unit 100A and the body fat measurement device 1A equipped with the same according to the present embodiment, the wrapping length of the bioelectrical impedance measurement abdomen attachment unit 100A changes following the breathing motion of the subject by appropriately adjusting the elastic force of the protrusion spring serving as the first biasing portion and the second biasing portion, and thus the subject does not feel an excessive oppressing feeling, and a bioelectrical impedance measurement abdomen attachment unit that is not painful to the subject can be obtained.

Therefore, the bioelectrical impedance measurement attachment unit which enables the electrode to be pressed against the body of the subject with a constant load in the attached state and which is not painful to the subject is realized with the bioelectrical impedance measurement abdomen attachment unit 100A according to the present embodiment. The body fat measurement device capable of calculating the body fat mass at high accuracy is realized with the body fat measurement device 1A equipped with the bioelectrical impedance measurement abdomen attachment unit 100A. Furthermore, the bioelectrical impedance measurement abdomen attachment unit and the body fat measurement device equipped with the same that enable the attachment task to be very easily performed are obtained.

In the following, a variant of the bioelectrical impedance measurement abdomen attachment unit according to the present invention will be described. The bioelectrical impedance measurement abdomen attachment unit according to first to third variants described below has a configuration similar to the bioelectrical impedance measurement abdomen attachment unit 100A of the present embodiment other than the portion particularly described. Thus, in the figure, the same reference numerals are denoted for the same or corresponding portions, and the description thereof will not be repeated.

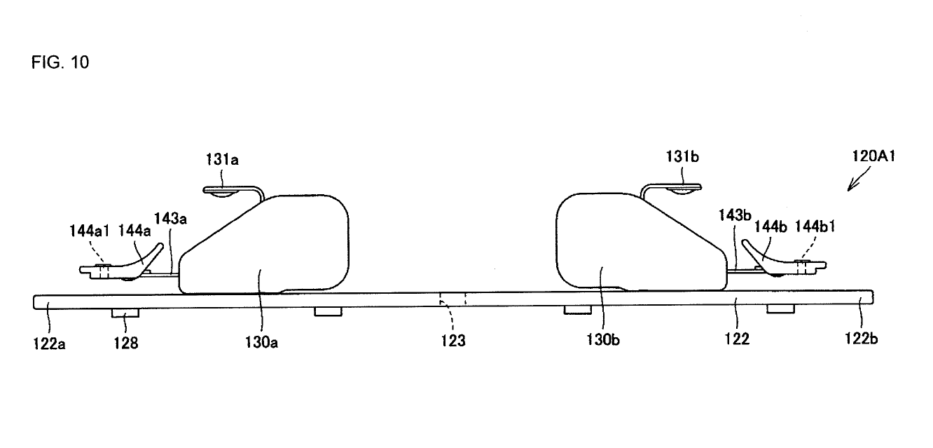

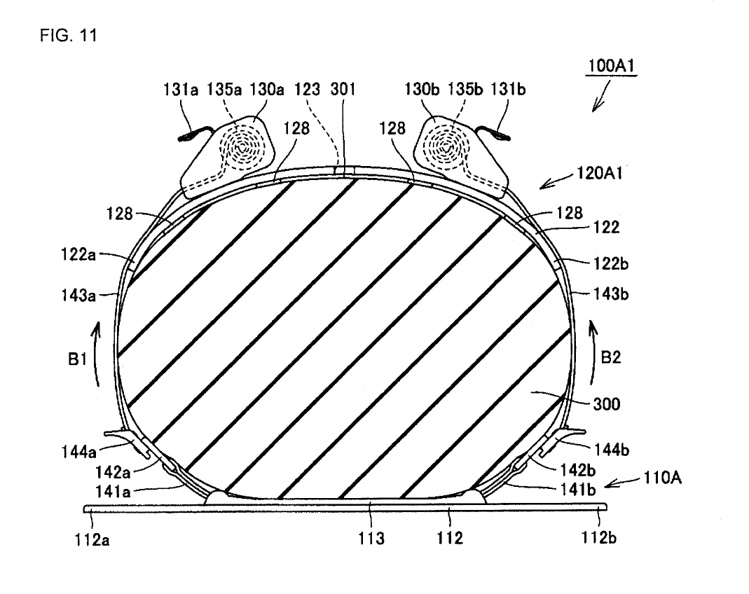

FIG. 10 is a side view of the abdomen front surface side base portion of the bioelectrical impedance measurement abdomen attachment unit according to a first variant of the present embodiment. FIG. 11 is a cross-sectional view showing a state in which the bioelectrical impedance measurement abdomen attachment unit according to the first variant is attached to the abdomen of the subject.

As shown in FIGS. 10 and 11, an abdomen front surface side base portion 120A1 of the bioelectrical impedance measurement abdomen attachment unit 100A1 according to the first variant includes an electrode 128 on the lower surface of the sheet 122. In other words, in the bioelectrical impedance measurement abdomen attachment unit 100A according to the above embodiment, the abdomen front surface side base portion has a divided configuration of the sheet 122 arranged with the wind-up units 130a, 130b, and the electrode film 126 arranged with the electrode 128, but in the bioelectrical impedance measurement abdomen attachment unit 100A1 according to the first variant, the wind-up units 130a, 130b and the electrode 128 are arranged on the sheet 122. According to such a configuration, the bioelectrical impedance measurement abdomen attachment unit that can be repeatedly used is obtained.

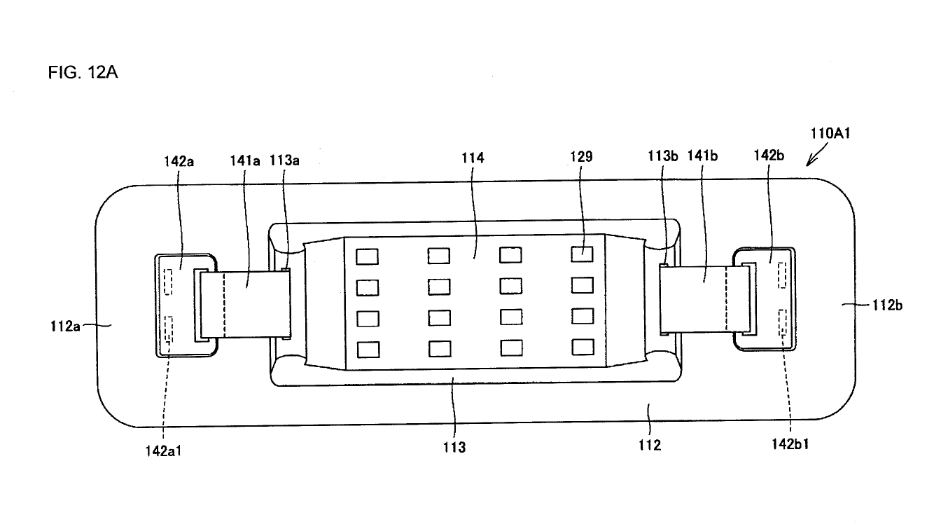

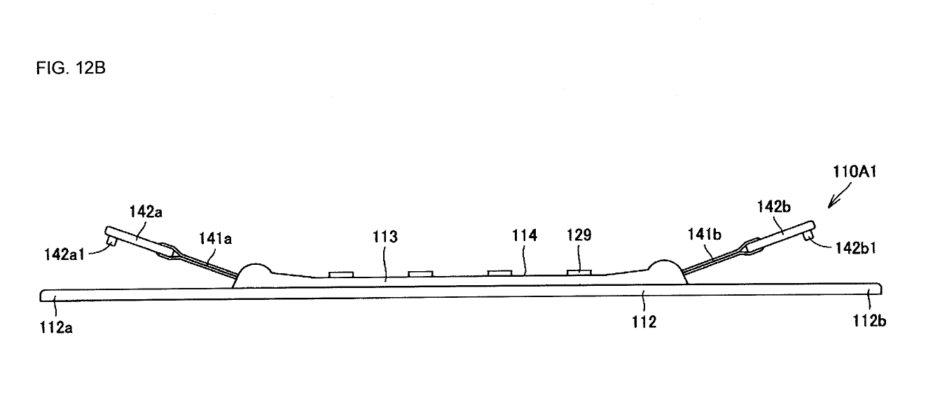

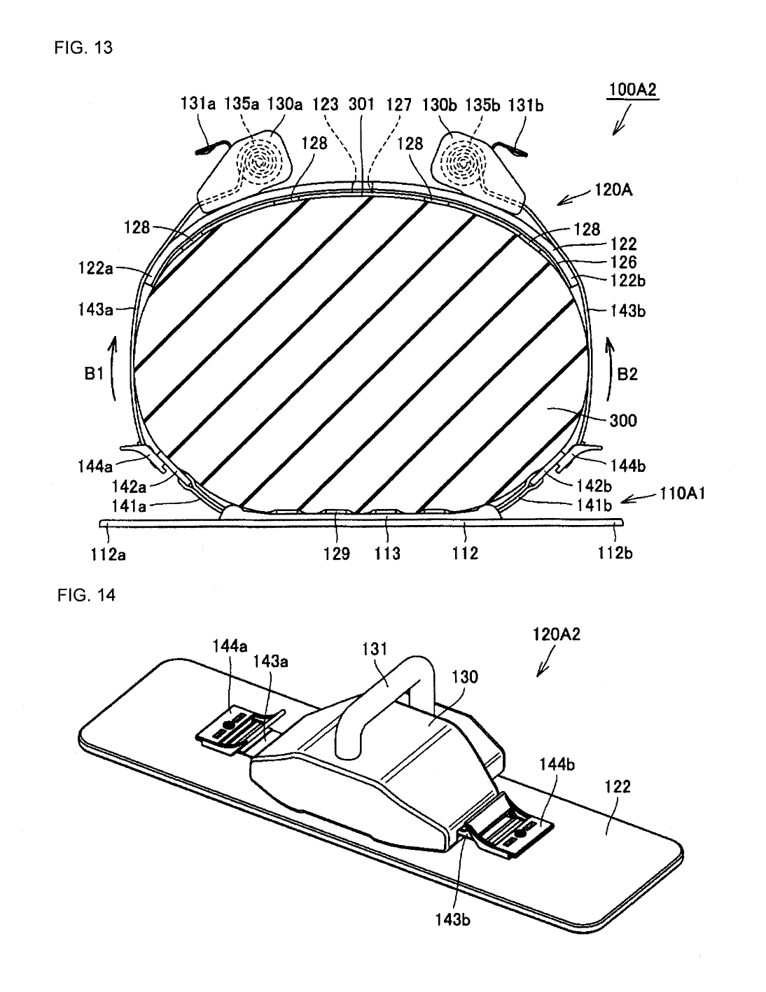

FIG. 12A is a top view of the abdomen rear surface side base portion of the bioelectrical impedance measurement abdomen attachment unit according to a second variant of the present embodiment, and FIG. 12B is a side view. FIG. 13 is a cross-sectional view showing a state in which the bioelectrical impedance measurement abdomen attachment unit according to the second variant is attached to the abdomen of the subject.

As shown in FIGS. 12A, 12B, and 13, the bioelectrical impedance measurement abdomen attachment unit 100A2 according to the second variant includes an electrode 129 on the abdomen rear surface side base portion 110A1 in addition to the electrode 128 arranged on the abdomen front surface side base portion 120A. More specifically, the electrode 129 is arranged on the upper surface 114 of the projection 113 of the abdomen rear surface side base portion 110A1, so that the electrode 129 comes into contact with the body rear surface of the subject when the subject lies down face up so as to cross the abdomen rear surface side base portion 110A1. According to such a configuration, the impedance measurement electrode is arranged in contact with not only the abdomen front surface but also the abdomen rear surface, and thus the body fat measurement of higher accuracy can be carried out. In another mode, the electrode arranged on the abdomen front surface side base portion may be eliminated and the electrode may be arranged only on the abdomen rear surface side base portion.

FIG. 14 is a perspective view of the abdomen front surface side base portion of the bioelectrical impedance measurement abdomen attachment unit according to the third variant of the present embodiment. As shown in FIG. 14, in the abdomen front surface side base portion 120A2 of the bioelectrical impedance measurement abdomen attachment unit according to the third variant, the wind-up unit 130 arranged on the sheet 122 is configured as one unit, and two wind-up mechanisms (not shown) are arranged inside the wind-up unit 130. An opening is formed at both end faces of the wind-up unit 130, and a pair of abdomen front surface side belt members 143a, 143b is pulled out from the openings. The grip portion 131 is formed substantially at the central part on the upper part of the wind-up unit 130. According to such a configuration, there is obtained the bioelectrical impedance measurement abdomen attachment unit, which device configuration is simplified than the bioelectrical impedance measurement abdomen attachment unit 100A according to the present embodiment.

### Second Embodiment

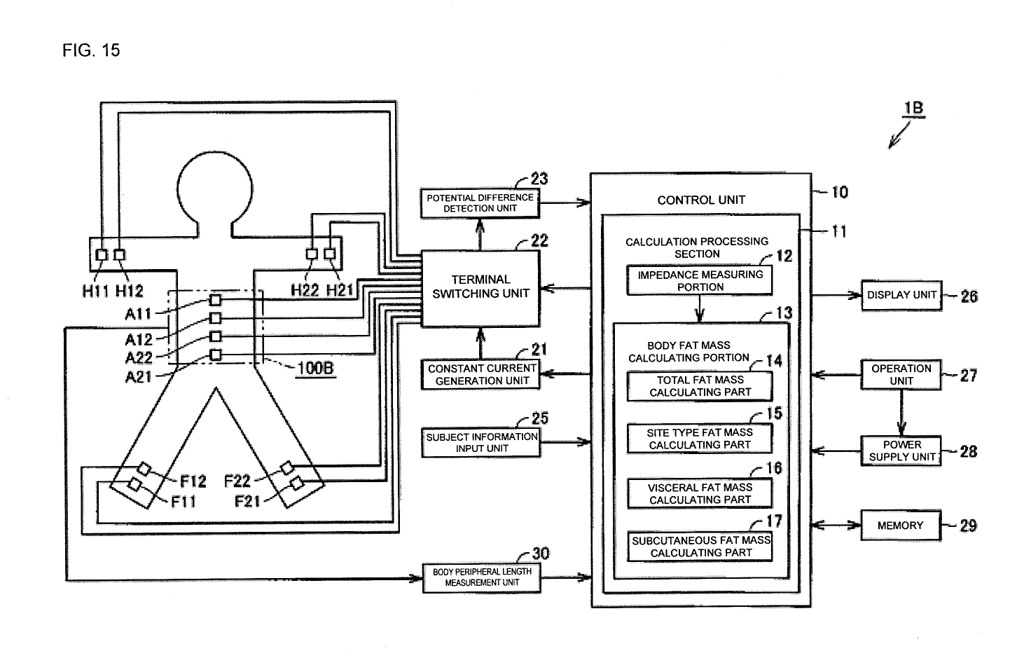

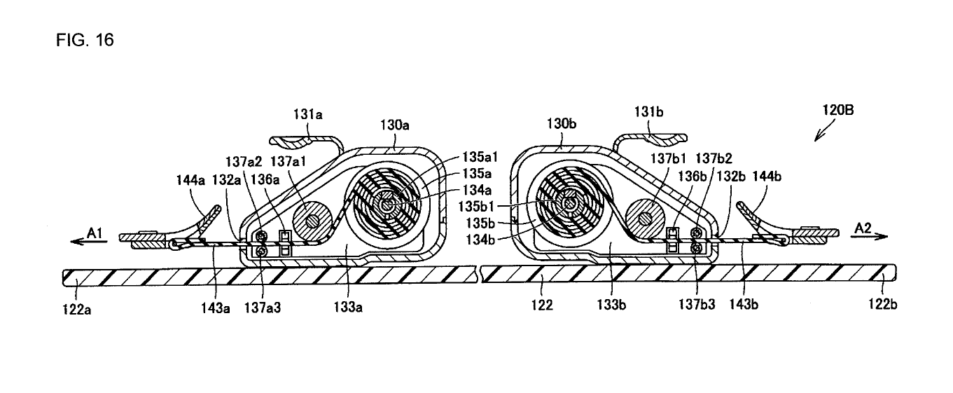

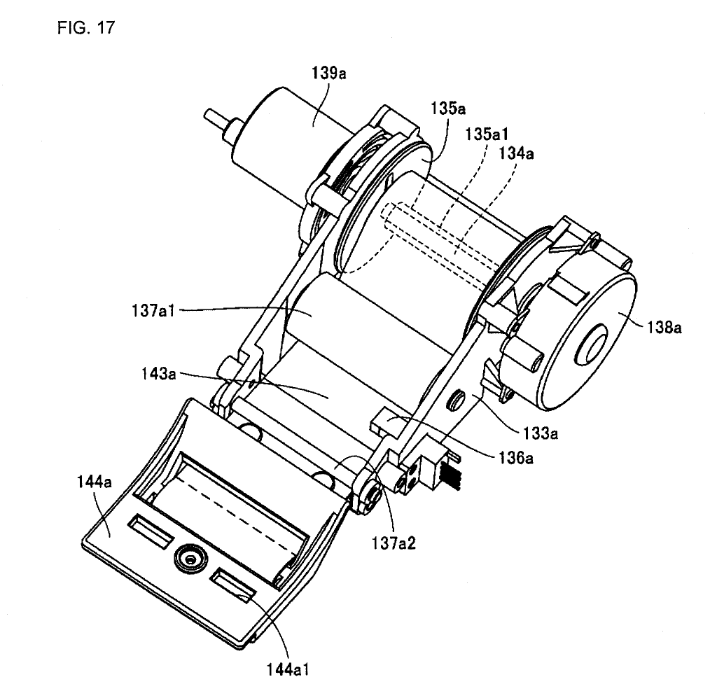

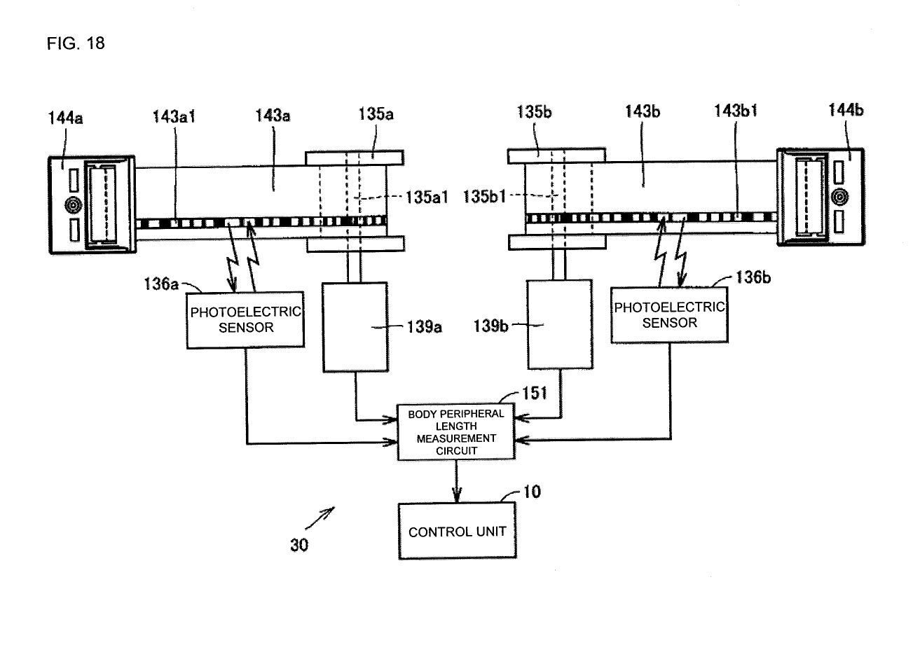

FIG. 15 is a function block diagram of a body fat measurement device according to a second embodiment of the present invention. FIG. 16 is a schematic cross-sectional view of a portion excluding the electrode film of the abdomen side base portion of the bioelectrical impedance measurement abdomen attachment unit equipped in the body fat measurement device according to the present embodiment, and FIG. 17 is a perspective view of the wind-up mechanism arranged in the wind-up unit of the abdomen front surface side base portion. Furthermore, FIG. 18 is a schematic view describing a configuration of a body peripheral length measurement unit of the body fat measurement device according to the present embodiment and a mechanism for automatically measuring the body peripheral length of the subject with the body peripheral length measurement unit. In the figure, the same reference numerals are denoted for portions similar to the first embodiment, and the description thereof will not be repeated.

As shown in FIG. 15, the body fat measurement device 1B according to the present embodiment includes a body peripheral length measurement unit 30 serving as a physical information measurement unit. The body peripheral length measurement unit 30 is a site for automatically measuring the body peripheral length of the subject, and measures the body peripheral length of the subject based on the output of various types of sensors arranged in the bioelectrical impedance measurement abdomen attachment unit 100B. A more specific configuration of the body peripheral length measurement unit 30 will be described below.

As shown in FIGS. 16 and 17, the wind-up mechanism arranged in the wind-up units 130a, 130b of the bioelectrical impedance measurement abdomen attachment unit 100B according to the present embodiment includes photoelectric sensors (more specifically, photo-interrupters) 136a, 136b. The photoelectric sensor 136a, 136b is assembled to the chassis 133a, 133b to face the main surface of the abdomen front surface side belt member 143a, 143b of the portion pulled out from the reel 135a, 135b. Thus, the abdomen front surface side belt member 143a, 143b pulled out from the reel 135a, 135b passes the lower side of the photoelectric sensor 136a, 136b. The photoelectric sensor 136a, 136b is one of the various types of sensors that configure the body peripheral length measurement unit 30, where the details thereof on the functions, operations, and the like will be described below.

As shown in FIG. 17, the wind-up mechanism arranged in the wind-up unit 130a includes a rotary encoder 139a. The rotary encoder 139a is assembled to the chassis 133a to be arranged at the side of the reel 135a, and the detection shaft is fixed to the shaft 135a1 of the reel 135a. Thus, the detection shaft of the rotary encoder 139a rotates with the rotation of the reel 135a. A rotary encoder is also attached to the wind-up mechanism arranged in the wind-up unit 130b, similar to the wind-up mechanism arranged in the wind-up unit 130a. Such a rotary encoder is one of the various types of sensors that configure the body peripheral length measurement unit 30, where the details thereof on the functions, operations, and the like will be described below.

As shown in FIG. 18, in the body fat measurement device 1B according to the present embodiment, the body peripheral length measurement unit 30 includes the photoelectric sensors 136a, 136b, the rotary encoders 139a, 139b, and the body peripheral length measurement circuit 151. An encoder strip 143a1, 143b1 is attached to the surface of the pair of abdomen front surface side belt members 143a, 143b configured to be wound up by the reel 135a, 135b. The encoder strip 143a1, 143b1 is arranged to extend from one end to the other end of the abdomen front surface side belt member 143a, 143b, where an identifier (barcode herein) indicating a position at the relevant portion of the abdomen front surface side belt member 143a, 143b is provided on the surface. The encoder strip 143a1, 143b1 is arranged facing the photoelectric sensor 136a, 136b in the wind-up unit 130a, 130b.

The photoelectric sensor 136a, 136b includes a light emitting portion and a light receiving portion, where the light emitted from the light emitting portion is radiated on the encoder strip 143a1, 143b1, and the reflected light is received by the light receiving portion. The photoelectric sensor 136a, 136b photoelectrically converts the received light to output an electrical signal, and inputs the same to the body peripheral length measurement circuit 151. The body peripheral length measurement circuit 151 detects the position of the abdomen front surface side belt member 143a, 143b of the portion arranged facing the photoelectric sensor 136a, 136b based on the input electrical signal.

The rotary encoder 139a, 139b detects the rotation angle of the shaft 135a1, 135b1 that rotates when the abdomen front surface side belt member 143a, 143b is sent from the reel 135a, 135b by the rotation of the detection shaft. The rotary encoder 139a, 139b outputs the electrical signal corresponding to the detected rotation angle, and inputs the same to the body peripheral length measurement circuit 151. The body peripheral length measurement circuit 151 detects the sent amount of the abdomen front surface side belt members 143a, 143b based on the input electrical signal.

The body peripheral length measurement circuit 151 detects the wrapping length of the bioelectrical impedance measurement abdomen attachment unit 1008 wrapped around the abdomen of the subject based on the positional information detected based on the electrical signal inputted from the photoelectric sensors 136a, 136b and the information on the sent amount detected based on the electrical signals inputted from the rotary encoders 139a, 139b, and outputs the same to the control unit 10.

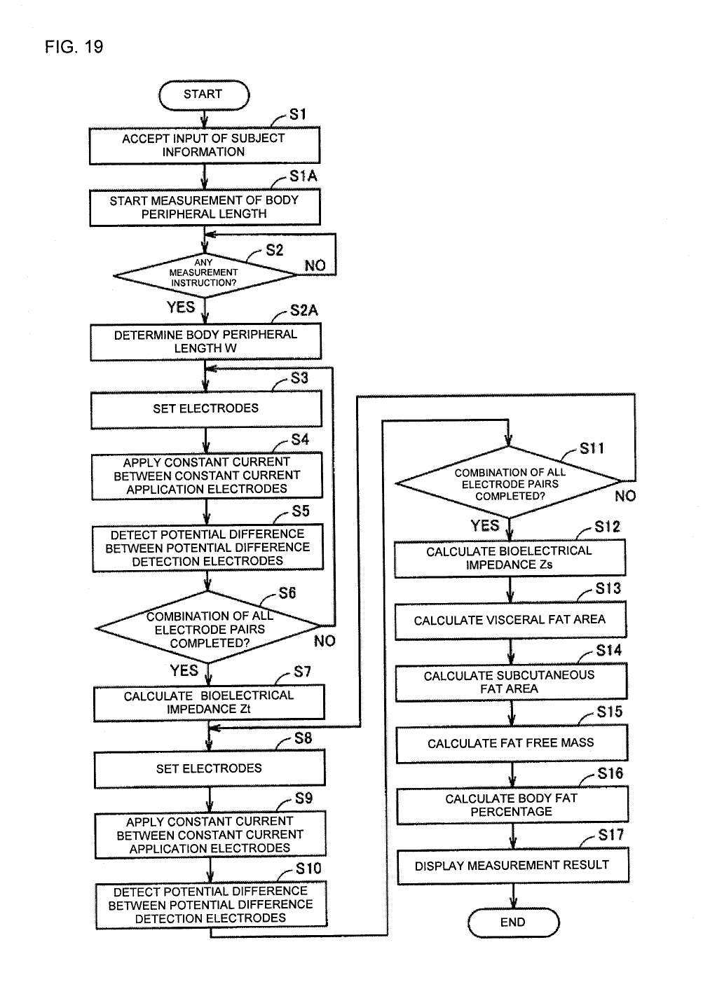

FIG. 19 is a flowchart defining the operation procedures of the body fat measurement device when measuring the visceral fat area, the subcutaneous fat area, and the body fat percentage using the body fat measurement device according to the present embodiment. The same step numbers are denoted for the steps similar to the first embodiment in the figure, and the detailed description thereof will not be repeated herein.

With reference to FIG. 19, the control unit 10 accepts the input of subject information including the height H and the weight Wt as physical information other than the body peripheral length W (step S1). The accepted subject information is temporarily saved in the memory 29.

The control unit 10 then starts the measurement operation of the body peripheral length (step S1A). Specifically, the photoelectric sensors 136a, 136b and the rotary encoders 139a, 139b start to operate based on a command of the control unit 10, and the impedance measurement abdomen attachment unit 100B is attached to the subject while the photoelectric sensors 136a, 136b and the rotary encoders 139a, 139b are operating.

The control unit 10 then determines whether or not an instruction to start the measurement is made (step S2). The control unit 10 waits until the instruction to start the measurement is made (NO in step S2), and when detecting the instruction to start the measurement (YES in step S2), the control unit 10 determines the information of the body peripheral length detected in the relevant state as the body peripheral length W of the subject (step 2A). The determined body peripheral length W is, for example, temporarily saved in the memory 29.

Step S12 is performed from step S3 similar to the step shown in the first embodiment, and the bioelectrical impedances Zt, Zs are calculated.

The visceral fat mass calculating part 16 then calculates the visceral fat area Sv based on the body peripheral length W specified in step S2A, and the calculated bioelectrical impedance Zt and the bioelectrical impedance Zs (step S13). The visceral fat area Sv is calculated by substituting the body peripheral length W and the calculated bioelectrical impedances Zt, Zs to equation (1).

The subcutaneous fat mass calculating part 17 calculates the subcutaneous fat area Ss based on the body peripheral length W specified in step S2A, and the calculated bioelectrical impedance Zs (step S14). The subcutaneous fat area Ss is calculated by substituting the body peripheral length W and the calculated bioelectrical impedance Zs to equation (2).

Thereafter, step S15 and step S16 similar to the steps shown in the first embodiment are performed, and the body fat percentage is calculated. The display unit 26 displays each measurement result based on the control of the control unit 10 (step S17).

The body fat measurement device 1B then ends the body fat mass measurement process including the visceral fat area measurement process, the subcutaneous fat area measurement process, and the body fat percentage measurement process.

According to the body fat measurement device 1B of the present embodiment described above, the body peripheral length of the subject can be automatically measured by the body peripheral length measurement unit 30 including the photoelectric sensors 136a, 136b and the rotary encoders 139a, 139b. Thus, the body peripheral length of the subject is easily obtained by simply attaching the bioelectrical impedance measurement abdomen attachment unit 100B, and the obtained body peripheral length can be used to calculate the body fat mass to thereby perform a highly accurate body fat measurement. The photoelectric sensor and the rotary encoder is generally very small, and thus a compact body fat measurement device capable of easily and automatically measuring the body peripheral length is obtained by adopting the above configuration. The barcode serving as a marker indicating the positional information may be easily configured by attaching the encoder strip 143a1, 143b1 to the pair of abdomen front surface side belt members 143a, 143b, whereby the device configuration does not become complicating and the device does not enlarge.

### Third Embodiment

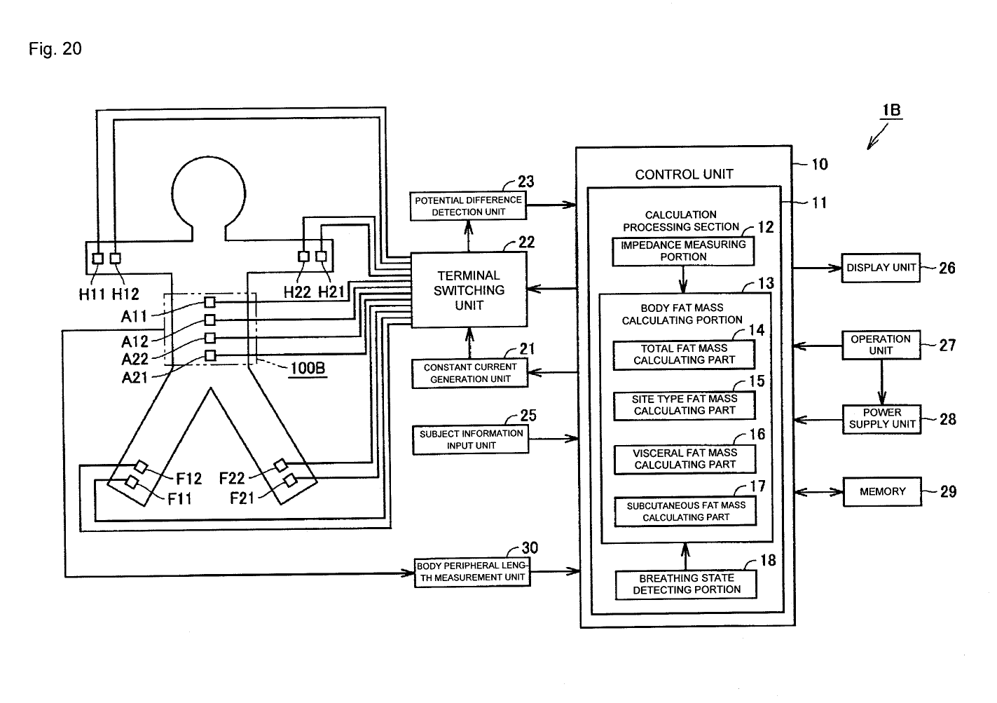

FIG. 20 is a view showing function blocks of a body fat measurement device according to a third embodiment of the present invention. First, with reference to FIG. 20, the configuration of the function blocks of the body fat measurement device 1C according to the present embodiment will be described. Same reference numerals are denoted for the portions similar to the second embodiment, and the description thereof will not be repeated herein.

As shown in FIG. 20, a body fat measurement device 1C according to the present embodiment includes a body peripheral length measurement unit 30 serving as a physical information measurement unit, similar to the body fat measurement device 1B according to the second embodiment. The body peripheral length measurement unit 30 is a unit for automatically measuring the body peripheral length of the subject, and measures the body peripheral length of the subject based on the output of various types of sensors arranged in the bioelectrical impedance measurement abdomen attachment unit 100C. The configuration of the body peripheral length measurement unit 30 is basically similar to the body fat measurement device 1B according to the second embodiment, and the body peripheral length measurement unit 30 includes the photoelectric sensors 136a, 136b, the rotary encoders 139a, 139b, and the body peripheral length measurement circuit 151 as shown in FIG. 18.

The body peripheral length of the subject constantly fluctuates, although slightly, with the breathing motion. The body peripheral length measurement unit 30 in the body fat measurement device 1C according to the present embodiment constantly measures the fluctuating body peripheral length during the measurement. The body peripheral length measurement unit 30 measures the body peripheral length of the subject by detecting the wrapping length of the bioelectrical impedance measurement abdomen attachment unit 100C wrapped around the abdomen of the subject and measures the fluctuation of the body peripheral length of the subject by detecting the fluctuation of the wrapping length of the bioelectrical impedance measurement abdomen attachment unit 100C wrapped around the abdomen of the subject. The body peripheral length measurement unit 30 outputs the information on the measured body peripheral length and the information of the fluctuation thereof to the control unit 10.

More specifically, the body peripheral length measurement circuit 151 specifies the body peripheral length of the subject based on the electrical signals inputted from the photoelectric sensors 136a, 136b, and outputs the same to the control unit 10. The body peripheral length measurement circuit 151 specifies the fluctuation of the body peripheral length of the subject based on the electrical signals inputted from the rotary encoders 139a, 139b, and outputs the same to the control unit 10.

In the body fat measurement device 1C according to the present embodiment, the calculation processing section 11 includes a breathing state detecting portion 18 in addition to the impedance measuring portion 12 and the body fat mass calculating portion 13. The breathing state detecting portion 18 detects the breathing state of the subject during the measurement operation based on the information of the body peripheral length of the subject measured by the body peripheral length measurement unit 30 and inputted to the control unit 10. The body fat mass calculating portion 13 calculates the body fat mass based on the bioelectrical impedance obtained in the impedance measuring portion 12, the information of the breathing state obtained in the breathing state detecting portion 18, the body peripheral length obtained in the body peripheral length measurement unit 30, and the subject information inputted from the subject information input unit 25.

In the present embodiment, the body peripheral length of the subject is specified based on the information detected by the photoelectric sensors 136a, 136b, and the fluctuation amount of the body peripheral length of the subject is specified based on the information detected by the rotary encoders 139a, 139b. However, the information outputted by the rotary encoders 139a, 139b may be used for the specification of the body peripheral length of the subject, or the information detected by the photoelectric sensors 136a, 136b may be used for the specification of the fluctuation amount of the body peripheral length of the subject.

One example of a calculation process performed by the body fat measurement device 1C according to the present embodiment will now be described. In the body fat measurement device 1C according to the present embodiment as well, the calculation process basically similar to that of the body fat measurement device 1A according to the first embodiment is performed, but the body fat measurement device 1C differs in that the value of the body peripheral length actually measured by the body peripheral length measurement unit 30 is used for the value of the body peripheral length W, and the values of the bioelectrical impedances Zt, Zs obtained in association with the information of the breathing state detected by the breathing state detecting portion 18 are used for the values of the bioelectrical impedances Zt, Zs used in various types of calculation processes.

The impedance measuring portion 12 calculates two types of bioelectrical impedances Zt, Zs based on the current value of the constant current generated by the constant current generation unit 21 and the potential difference detected by the potential difference detection unit 23, but the bioelectrical impedance Zt that reflects the fat free mass at the abdomen of the subject and the bioelectrical impedance Zs that reflects the subcutaneous fat mass at the abdomen of the subject both vary from hour to hour according to the breathing motion of the subject.

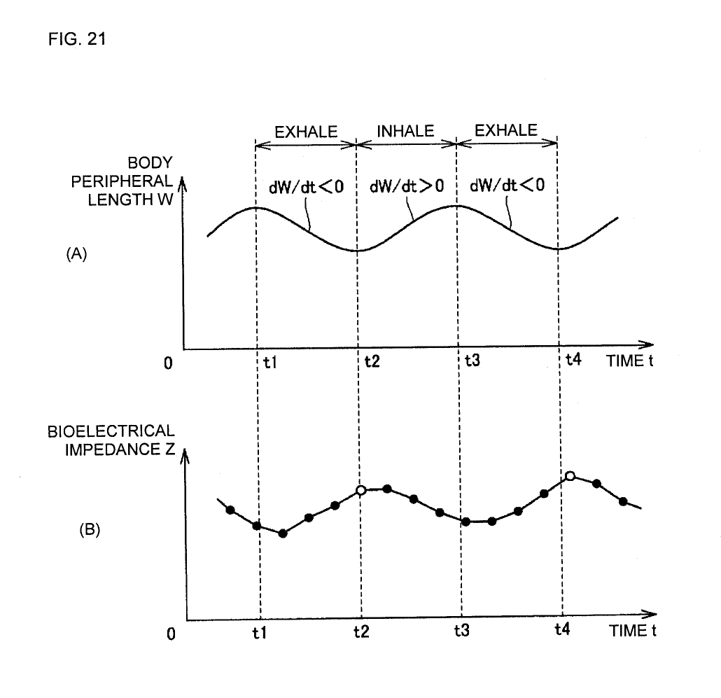

FIG. 21 is a graph showing a relationship of the fluctuation of the body peripheral length of the subject and the bioelectrical impedance that varies from hour to hour. In FIG. 21, a horizontal axis indicates time, where a vertical axis of a portion (A) indicates the body peripheral length and a vertical axis of a portion (B) indicates the bioelectrical impedance.

As shown in the portion (A) of FIG. 21, the body peripheral length W of the subject fluctuates in accordance with the breathing motion of the subject, where the body peripheral length W increases when the subject performs the inhaling motion and the body peripheral length W decrease when the subject performs the exhaling motion. On the contrary, the bioelectrical impedance Z also fluctuates in accordance with the breathing motion of the subject as shown in the portion (B) of FIG. 21, where the value generally decreases when the subject performs the inhaling motion and the value generally increases when the subject performs the exhaling motion.

In the body fat measurement device 1C according to the present embodiment, the following processes are performed on the acquired data to exclude such a fluctuation involved in the breathing motion of the bioelectrical impedance Z as an error component. First, for a predetermined period defined in advance, the potential difference between the potential difference detection electrodes is measured by the potential difference detection unit 23 over a plurality of times at a predetermined interval, and the data of the obtained potential difference is acquired as time-series data. The time-series data of the bioelectrical impedance Z is then obtained from the time-series data of the potential difference obtained by the impedance measuring portion 12. In parallel thereto, the body peripheral length W of the subject of a period same as the period in which the detection of the potential difference is carried out is acquired as time-series data by the body peripheral length measurement unit 30.

The time-series data of the acquired bioelectrical impedance Z and the time-series data of the body peripheral length W are then synchronized. Thereafter, dW/dt at each time is calculated based on the time-series data of the body peripheral length Win the breathing state detecting portion 18. If the calculated dW/dt takes a positive value (i.e., dW/dt>0), the subject is determined as performing the exhaling motion (e.g., period of t2 to t3 shown in the portion (A) of FIG. 21), whereas if the calculated dW/dt takes a negative value (i.e., dW/dt<0), the subject is determined as performing the inhaling motion (e.g., period of t1 to t2, t3 to t4 shown in the portion (A) of FIG. 21). The time of transitioning from the exhaling motion to the inhaling motion (i.e., time at which dW/dt=0, or time at which dW/dt changes from a negative value to a positive value) is then specified (e.g., time t2, t4 shown in the portion (A) of FIG. 21).

The bioelectrical impedance (e.g., bioelectrical impedance shown with an outlined circle in the portion (B) of FIG. 21) acquired at the time closest to or the time same as the time of transitioning from the exhaling motion to the inhaling motion is extracted from the time-series data of the bioelectrical impedance Z, and the average value of the extracted data is decided as a representative value of the bioelectrical impedance Z. The average value of the body peripheral length acquired at the time closest to or the time same as the time of transitioning from the exhaling motion to the inhaling motion is decided as a representative value of the body peripheral length W of the subject.

The method of deciding the representative value of the bioelectrical impedance described above merely shows one example. A case of using the bioelectrical impedance acquired at the timing of transitioning from the exhaling motion to the inhaling motion as the representative value has been shown, but the bioelectrical impedance acquired at the timing of transitioning from the inhaling motion to the exhaling motion may be used as the representative value. Instead of simply extracting specific data from the time-series data of the bioelectrical impedance Z and obtaining the average value thereof to decide the representative value, other calculations or the like may be added to decide the representative value. In either case, the representative value of the bioelectrical impedance Z merely needs to be decided in association with the breathing motion of the subject detected from the fluctuation of the body peripheral length of the subject.

In the body fat measurement device 1C according to the present embodiment, various types of fat mass are calculated using the representative value of the body peripheral length W and the representative values of the bioelectrical impedances Zt, Zs obtained in the above manner. Equations (1) to (4) shown in the first embodiment are used for the equations for calculating the same.

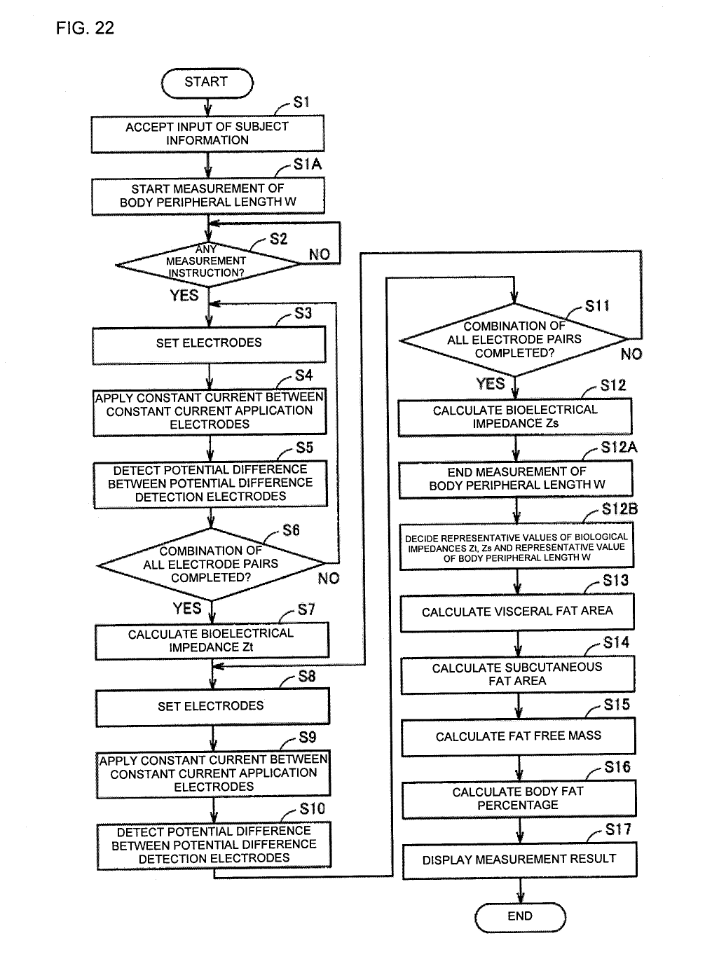

FIG. 22 is a flowchart showing the operation procedures of the body fat measurement device in measuring the visceral fat area, the subcutaneous fat area, and the body fat percentage using the body fat measurement device according to the present embodiment. In the figure, the same step numbers are denoted for the steps similar to the first embodiment, and the detailed description thereof will not be repeated herein.

With reference to FIG. 22, the control unit 10 accepts the input of the height H, the weight Wt and the like as physical information other than the body peripheral length W (step S1). The accepted subject information is temporarily saved in the memory 29.

The control unit 10 then outputs a command to start the measurement of the body peripheral length to the body peripheral length measurement unit 30, and the body peripheral length measurement unit 30 starts the measurement of the body peripheral length W based thereon (step S1A).

The control unit 10 determines whether or not an instruction to start the measurement is made (step S2). The control unit 10 waits until the instruction to start the measurement is made (NO in step S2). The control unit 10 proceeds to step S3 if the instruction to start the measurement is detected (YES in step S2).

The control unit 10 then sets the electrode (step S3), and the constant current generation unit 21 flows a constant current between the upper limb and the lower limb based on the control of the control unit 10 (step S4). In this state, the potential difference detection unit 23 detects the potential difference between the abdominal electrodes serving as the selected potential difference detection electrode over a plurality of times at a predetermined interval for a predetermined period defined in advance based on the control of the control unit 10 (step S5).

The control unit 10 determines whether or not the detection of the potential difference is completed for the combinations of all abdominal electrode pairs serving as the potential difference detection electrode defined in advance (step S6). The control unit 10 proceeds to the process of step S3 if determined that the detection of the potential difference is not completed for the combination of all of the abdominal electrode pairs serving as the potential difference detection electrode defined in advance (NO in step S6), and selects the non-selected abdominal electrode pair. The control unit 10 thereby detects, in order, the potential difference between the abdominal electrodes of the potential difference detection electrode pair of a plurality of pairs.

When the detection of the potential difference is completed for the combination of the abdominal electrode pairs serving as the potential difference detection electrode pair defined in advance (YES in step S6), the impedance measuring portion 12 calculates the time-series data of the bioelectrical impedances Zt1 to Zt4 based on the current value of the constant current generated by the constant current generation unit 21 and flowed through the body and the time-series data of each potential difference detected by the potential difference detection unit 23 (step S7). The time-series data of the bioelectrical impedances Zt1 to Zt4 calculated by the impedance measuring portion 12 is associated with the time-series data of the body peripheral length W measured by the body peripheral length measurement unit 30, and temporarily saved in the memory 29.

The control unit 10 then sets the electrodes again (step S8), and the constant current generation unit 21 flows a constant current between the abdominal electrodes serving as the selected constant current application electrode based on the control of the control unit 10 (step S9). In this state, the potential difference detection unit 23 detects the potential difference between the abdominal electrodes serving as the selected potential difference detection electrode over a plurality of times at a predetermined interval for a predetermined period defined in advance based on the control of the control unit 10 (step S10).

The control unit 10 determines whether or not the application of the constant current and the detection of the potential difference are completed for all of the combinations of the constant current application electrode pairs and the potential difference detection electrode pairs defined in advance (step S11). The control unit 10 proceeds to the process of step S8 if determined that the application of the constant current and the detection of the potential difference are not completed for all of the combinations of the constant current application electrode pairs and the potential difference detection electrode pairs defined in advance (NO in step S11), and selects the non-selected electrode pair. In this manner, the control unit 10 performs constant current application and potential difference detection, in order, on all of the combinations of the constant current application electrode pair and the potential difference detection electrode pair defined in advance.

When the application of the constant current and the detection of the potential difference are completed for all of the combinations of the constant current application electrode pairs and the potential difference detection electrode pairs defined in advance (YES in step S11), the impedance measuring portion 12 calculates the time-series data of the bioelectrical impedances Zs1 to Zs4 based on the current value of the constant current generated by the constant current generation unit 21 and flowed through the body and the time-series data of each potential difference detected by the potential difference detection unit 23 (step S12). The time-series data of the bioelectrical impedances Zs1 to Zs4 calculated by the impedance measuring portion 12 is associated with the time-series data of the body peripheral length W measured by the body peripheral length measurement unit 30, and temporarily saved in the memory 29.

The control unit 10 then outputs a command to end the measurement of the body peripheral length to the body peripheral length measurement unit 30, and the body peripheral length measurement unit 30 ends the measurement of the body peripheral length W based thereon (step S12A). Thereafter, the body fat mass calculating portion 13 decides the representative values of the bioelectrical impedances Zt1 to Zt4 and the bioelectrical impedances Zs1 to Zs4 and decides the representative value of the body peripheral length W based on the time-series data of the bioelectrical impedances Zt1 to Zt4 and the time-series data of the bioelectrical impedances Zs1 to Zs4, which are temporarily saved in the memory 29 and associated with the times-series data of the body peripheral length W (step S12B). The method of deciding the representative value is as described above.

The visceral fat mass calculating part 16 then calculates the visceral fat area Sv based on the representative value of the actually measured body peripheral length W, the representative value of the calculated bioelectrical impedances Zt1 to Zt4, and the representative value of the bioelectrical impedances Zs1 to Zs4 (step S13). The visceral fat area Sv is calculated from equation (1). In the case where four sets of abdominal electrode groups, each set including four abdominal electrodes A11, A12, A21, A22, are arranged parallel to each other as mentioned above, the average value of the representative values of the four bioelectrical impedances Zt1 to Zt4 and the average value of the representative values of the four bioelectrical impedances Zs1 to Zs4, for example, are respectively substituted to equation (1).

The subcutaneous fat mass calculating part 17 then calculates the subcutaneous fat area Ss based on the representative value of the actually measured body peripheral length W, and the representative value of the calculated bioelectrical impedances Zs1 to Zs4 (step S14). The subcutaneous fat area Ss is calculated by substituting the body peripheral length W and the calculated bioelectrical impedance Zs to equation (2). In the case where four sets of abdominal electrode groups, each set including four abdominal electrodes A11, A12, A21, A22, are arranged parallel to each other as mentioned above, the average value of the representative values of the four bioelectrical impedances Zs1 to Zs4, for example, is substituted to the bioelectrical impedance Zs in equation (2).

The total fat mass calculating part 14 calculates the fat free mass FFM based on the height H of the physical information accepted by the control unit 10 in step S1 and the representative value of the calculated bioelectrical impedance Zt (step S15). The fat free mass FFM is calculated by equation (3). In the case where four sets of abdominal electrode groups, each set including four abdominal electrodes A11, A12, A21, A22, are arranged parallel to each other as mentioned above, the average value of the representative values of the four bioelectrical impedances Zt1 to Zt4, for example, is substituted to the bioelectrical impedance Zt in equation (3).

The total fat mass calculating part 14 calculates the body fat percentage based on the weight Wt of the physical information accepted by the control unit 10 in step S1, and the fat free mass FFM calculated by the total fat mass calculating part 14 in step S15 (step S16). The body fat percentage is calculated from equation (4).

The display unit 26 displays each measurement result based on the control of the control unit 10 (step S17).

The body fat measurement device 1B then ends the body fat mass measurement process including the visceral fat area measurement process, the subcutaneous fat area measurement process, and the body fat percentage measurement process.

The breathing state of the subject can be detected at high accuracy with a simple configuration of detecting the fluctuation of the wrapping length of the bioelectrical impedance measurement abdomen attachment unit 100C at the time of measurement by adopting the configuration of the body fat measurement device 1C according to the present embodiment described above. The change in the body peripheral length of the subject involved in the breathing motion can be captured at high accuracy by using such a detection method. Thus, the bioelectrical impedance can be accurately measured excluding the influence of the fluctuation of the bioelectrical impedance that occurs with the breathing motion by adopting the above detection method and acquiring the value of the bioelectrical impedance as the time-series data and determining the representative value of the bioelectrical impedance in association with the breathing motion of the subject. As a result, the body fat measurement device capable of measuring the body fat mass at high accuracy can be obtained. In particular, since the bioelectrical impedance is to be measured with the electrode arranged in contact with the abdomen of the subject in order to measure the visceral fat mass and the subcutaneous fat mass at the abdomen at high accuracy, the visceral fat mass and the subcutaneous fat mass at the abdomen can be particularly calculated at high accuracy with the body fat measurement device 1C of such a configuration.

### Fourth Embodiment

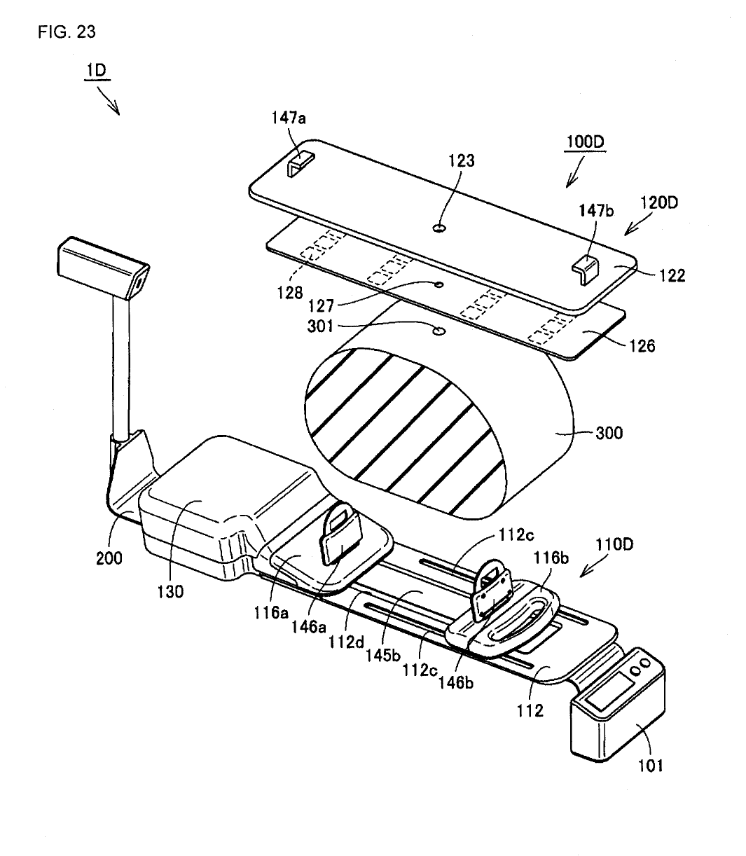

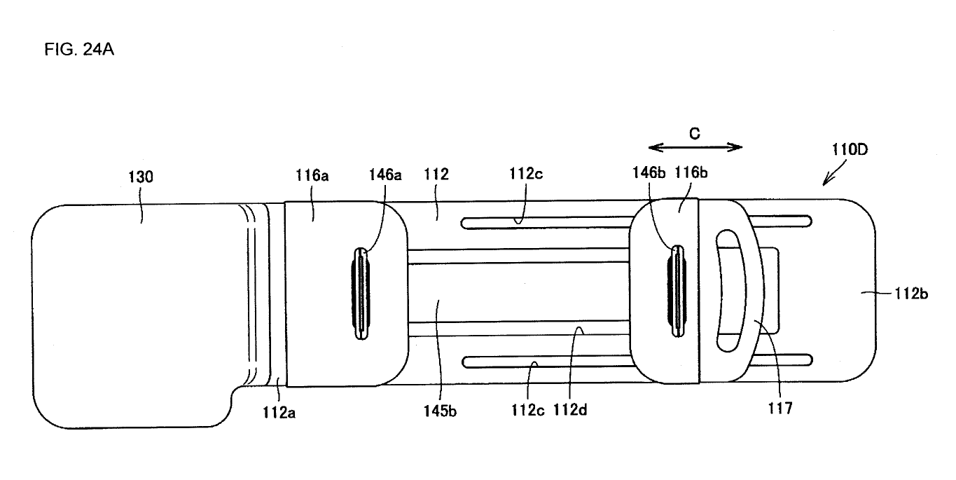

FIG. 23 is a schematic perspective view showing an outer appearance structure of a body fat measurement device according to a fourth embodiment of the present invention. First, the outer appearance structure of a body fat measurement device 1D according to the present embodiment will be described with reference to the drawing. The body fat measurement device 1D according to the present embodiment has exactly the same configuration as the body fat measurement device 16 according to the second embodiment in the function blocks, but the specific structure of the arranged bioelectrical impedance measurement abdomen attachment unit 100D differs from the bioelectrical impedance measurement abdomen attachment unit 1006 of the second embodiment. Therefore, same reference numerals are denoted for the same or corresponding portions, and the description thereof will not be repeated herein.

As shown in FIG. 23, the body fat measurement device 1D according to the present embodiment mainly includes a device main body 101, the bioelectrical impedance measurement abdomen attachment unit 100D, a pair of bioelectrical impedance measurement upper limb attachment units (not shown), and a pair of bioelectrical impedance measurement lower limb attachment units (not shown). The configuration of the device main body 101, the pair of bioelectrical impedance measurement upper limb attachment units and the pair of bioelectrical impedance measurement lower limb attachment units are exactly the same as the configuration according to the second embodiment.

The bioelectrical impedance measurement abdomen attachment unit 100D is aimed to be attached when the subject is lying down face up on the bed (not shown). The bioelectrical impedance measurement abdomen attachment unit 100D includes an abdomen rear surface side base portion 110D serving as a first base portion and an abdomen front surface side base portion 120D serving as a second base portion. The abdomen rear surface side base portion 110D is placed on the abdomen rear surface of the abdomen 300 of the subject in the attached state. The abdomen front surface side base portion 120D is placed on the abdomen front surface of the abdomen 300 of the subject in the attached state. More specifically, the abdomen rear surface side base portion 110D is attached to a bed with the attachment tool 200 in advance, and is placed on the abdomen rear surface of the subject in a state the subject is laid down in a face-up position on the bed surface. The abdomen front surface side base portion 120D is placed on the abdomen front surface of the subject by being mounted on the abdomen front surface of the subject in a state the subject is laid down in the face-up position on the bed surface. The abdomen front surface side base portion 120D includes the sheet 122 serving as a first sheet-like member and the electrode film 126 serving as a second sheet-like member.

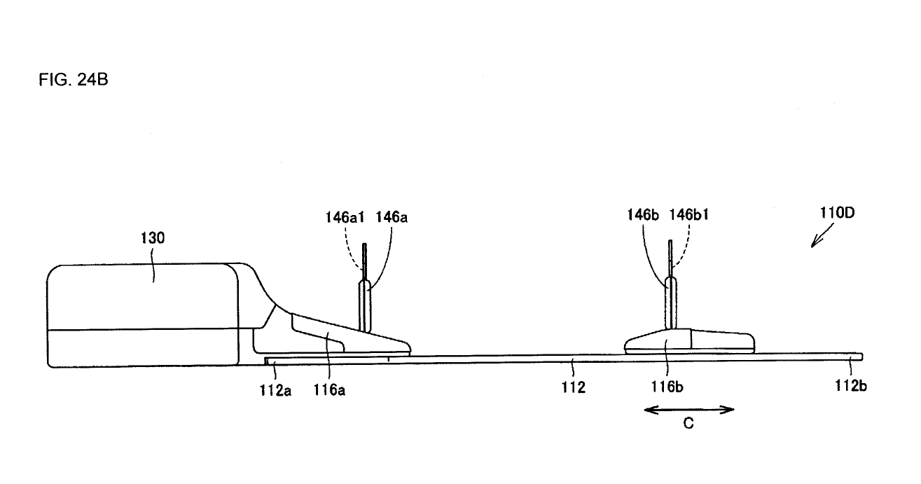

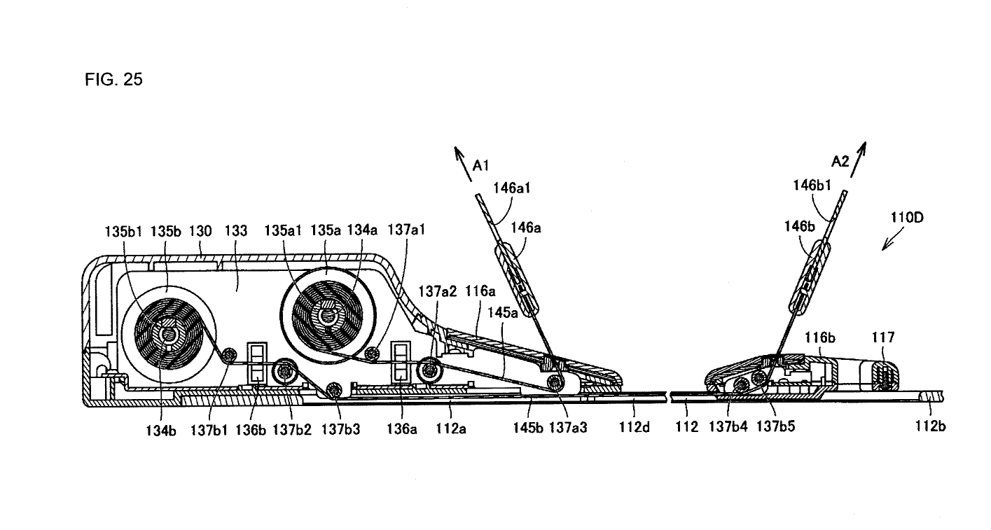

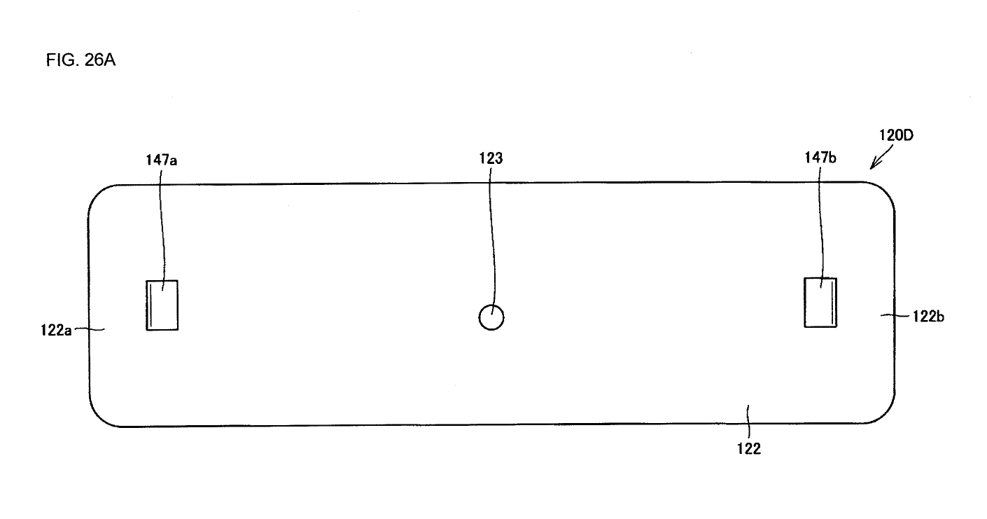

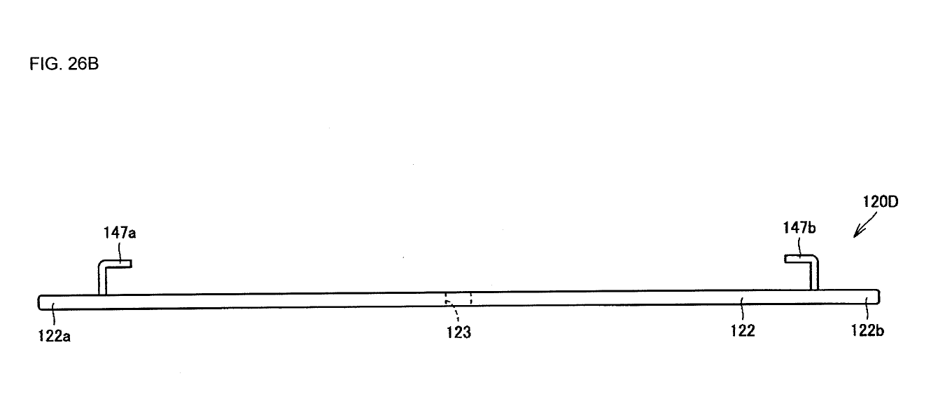

FIG. 24A is a top view of the abdomen rear surface side base portion of the bioelectrical impedance measurement abdomen attachment unit shown in FIG. 23, and FIG. 24B is a side view of the abdomen rear surface side base portion. FIG. 25 is a cross-sectional view of the abdomen rear surface side base portion of the bioelectrical impedance measurement abdomen attachment unit shown in FIG. 23. FIG. 26A is a top view of the abdomen front surface side base portion of the bioelectrical impedance measurement abdomen attachment unit shown in FIG. 23, and FIG. 26B is a side view of the portion excluding the electrode film of the abdomen front surface side base portion. In the following, the outer appearance structure of the bioelectrical impedance measurement abdomen attachment unit 100D according to the present embodiment will be described in more detail with such figures and FIG. 23.

As shown in FIGS. 23, 24A, 24B, and 25, the abdomen rear surface side base portion 110D has a substantially rectangular outer shape having the direction corresponding to the waistline direction of the subject as the longitudinal direction in the attached state. The abdomen rear surface side base portion 110D mainly includes a plate-shaped support base 112, guide units 116a, 116b arranged at a portion closer to one end 112a in the longitudinal direction of the support base 112 and a portion closer to the other end 112b, respectively, a pair of belt members 145a, 145b arranged in a manner capable of being pulled out from the guide units 116a, 116b, respectively, and a wind-up unit 130 arranged on the outer side than the one end 112a of the support base 112.

The support base 112 has the lower surface fixed to the attachment tool 200, and is configured such that the abdomen rear surface of the subject can be received at the portion between the guide units 116a, 116b of the upper surface.

One guide unit 116a is integrated with the wind-up unit 130, and is immovably fixed on the support base 112. The other guide unit 116b is configured as a separate body from the wind-up unit 130, and is movably attached on the support base 112. More specifically, the support base 112 is formed with a slit 112c extending in the longitudinal direction, where the guide unit 116b is guided to the slit 112c and is movable in the longitudinal direction (direction of arrow C shown in FIGS. 24A and 24B) on the support base 112 by fitting an engagement portion (not shown) arranged at the lower part of the guide unit 116b to the slit 112c. In other words, according to such a configuration, an interval adjustment mechanism for adjusting the interval between the guide unit 116a and the guide unit 116b according to the abdominal lateral width of the subject is configured. The guide unit 116b includes a grip portion 117 that can be gripped by hand in use.

The wind-up unit 130 interiorly includes a wind-up mechanism serving as a wind-up mechanism described below. The wind-up mechanism is fixed with one end of the pair of belt members 145a, 145b. The belt member 145a, which other end is pulled out from the guide unit 116a, of the pair of belt members 145a, 145b is guided by rollers 137a1, 137a2, 137a3 arranged inside the wind-up unit 130 and the guide unit 116a therethrough. The belt member 145b, which other end is pulled out from the guide unit 116b, of the pair of belt members 145a, 145b is guided through the wind-up unit 130 by rollers 137b1, 137b2, 137b3 arranged inside the wind-up unit 130, inserted through the slid 112d formed substantially at the middle of the support base 112, and guided through the guide unit 116 by rollers 137b4, 137b5 arranged inside the guide unit 116b. Connection tools 146a, 146b are respectively attached to the ends on the side pulled out from the guide units 116a, 116b of the pair of belt members 145a, 145b. Locking recesses 146a1, 146b1 are respectively arranged at predetermined positions of the connection tools 146a, 146b.

As shown in FIGS. 23, 26A, and 26B, the sheet 122 of the abdomen front surface side base portion 120D has a substantially rectangular outer shape having the direction corresponding to the waistline direction of the subject as the longitudinal direction in the attached state. The sheet 122 is made of a flexible material so as to deform along the shape of the abdomen front surface of the subject and fit to the abdomen front surface when placed on the abdomen front surface of the abdomen 300 of the subject. The positioning through-hole 123 serving as a mark for alignment with the umbilicus 301 of the subject in attachment is arranged substantially at the central part of the sheet 122. A lock projection 147a is arranged at the portion closer to one end 122a in the longitudinal direction of the sheet 122, and a lock projection 147b is arranged at the portion closer to the other end 122b in the longitudinal direction of the sheet 122. The lock projections 147a, 147b can engage with the lock recesses 146a, 146b1 formed in the connection tools 146a, 146b.

As shown in FIG. 23, the electrode film 126 of the abdomen front surface side base portion 120D has a substantially rectangular outer shape having the direction corresponding to the waistline direction of the subject as the longitudinal direction in the attached state, and a specific configuration thereof is exactly the same as the configuration described using FIGS. 6A and 6B in the first embodiment.

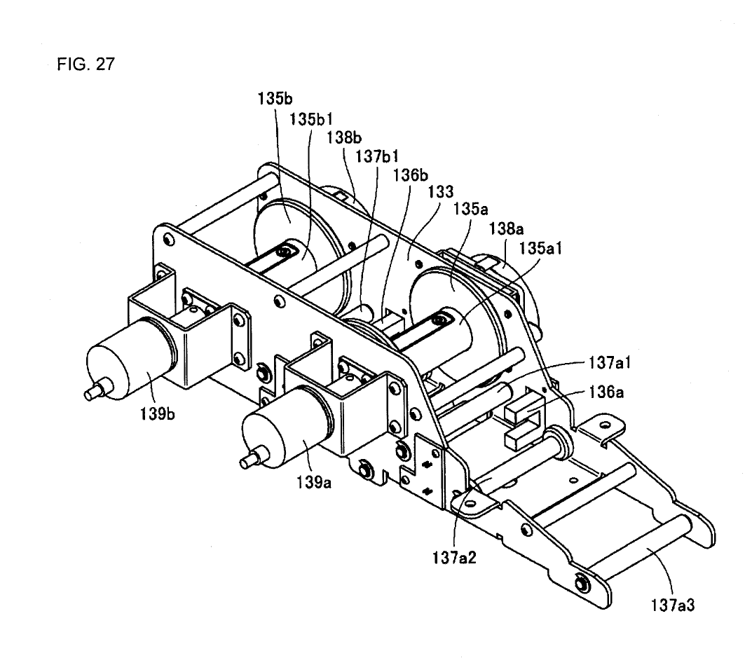

FIG. 27 is a perspective view of the wind-up mechanism arranged inside the wind-up unit. With reference to this figure and FIG. 25, a structure of the wind-up mechanism arranged in the abdomen rear surface side base portion 110D will be described in detail.

As shown in FIGS. 25 and 27, the chassis 133 is arranged inside the wind-up unit 130, and the pair of wind-up mechanisms is assembled to the chassis 133. Each of the pair of wind-up mechanisms is configured by a support shaft 134a, 134b, a reel 135a, 135b, a biasing portion 138a, 138b, and rollers 137a1, 137a2, and 137a3, 137b1, 137b2, 137b3. The support shaft 134a, 134b is axially supported by the chassis 133, and a shaft 135a, 135b11 of the reel 135a, 135b is externally inserted to the support shaft 134a, 134b. The reel 135a, 135b is fixed with one end of the belt member 145a, 145b, and the belt member 145a, 145b is wrapped around thereto. The biasing portion 138a, 138b interiorly accommodates a protrusion spring (not shown) serving as a first biasing portion and a second serving portion, which protrusion spring is connected to the reel 135a, 135b.

According to the wind-up mechanism of the above configuration, the pair of belt members 145a, 145b are configured to be able to be pulled out from the reels 135a, 135b towards the direction of arrows A1, A2 in FIG. 25, and the belt members 145a, 145b are wound up by the reels 135a, 135b by the elastic force of the protrusion spring serving as the first biasing portion and the second biasing portion when force is not applied on the belt members 145a, 145b.

As shown in FIGS. 25 and 27, in the bioelectrical impedance measurement abdomen attachment unit 100D according to the present embodiment as well, the photoelectric sensors 136a, 136b and the rotary encoders 139a, 139b configuring the body peripheral length measurement unit 30 are arranged in the chassis 133 arranged in the wind-up unit 130. The specific assembly structure of the photoelectric sensors 136a, 136b and the rotary encoders 139a, 139b is similar to that of the second embodiment.

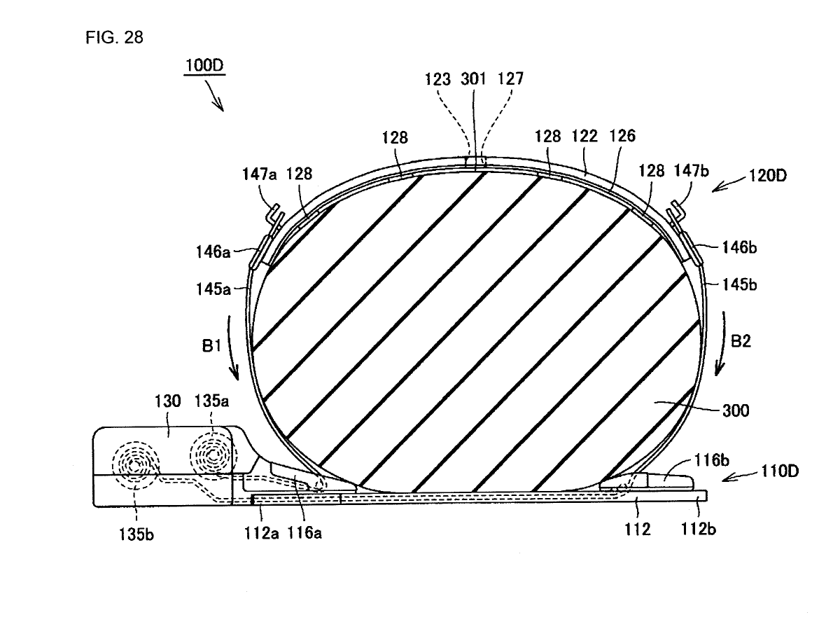

FIG. 28 is a schematic cross-sectional view showing a state in which the bioelectrical impedance measurement abdomen attachment unit of the above configuration is attached to the abdomen of the subject. The procedures for attaching the bioelectrical impedance measurement abdomen attachment unit 100D according to the present embodiment to the abdomen of the subject, and the attached state will be described below with reference to FIG. 28.

When attaching the bioelectrical impedance measurement abdomen attachment unit 100D according to the present embodiment, the subject lies down face up on the bed. In this case, the guide unit 116b is movement adjusted to the left and the right in accordance with the lateral width of the abdomen of the subject, and the abdomen 300 of the subject is positioned on the abdomen rear surface side base portion 110D arranged on the bed. The electrode film 126 is then mounted on the abdomen front surface of the subject. In this case, the alignment of the positioning through-hole 127 formed in the electrode film 126 and the umbilicus 301 of the subject is performed. The sheet 122 is then mounted on the electrode film 126 mounted on the abdomen front surface of the subject. In this case, the alignment of the positioning through-hole 123 formed in the sheet 122 and the umbilicus 301 of the subject is performed.

Subsequently, the belt members 145a, 145b are pulled out from the guide units 116a, 116b, respectively, and the connection tools 146a, 146b arranged at the distal ends are connected to the abdomen front surface side base portion 120D. Such connection is carried out by locking the lock projections 147a, 147b arranged on the abdomen front surface side base portion 120D to the lock recesses 146a1, 146b1 formed in the connection tools 146a, 146b. The attachment of the bioelectrical impedance measurement abdomen attachment unit 100D to the abdomen 300 of the subject is completed in the above manner.

As shown in FIG. 28, in a state the bioelectrical impedance measurement abdomen attachment unit 100D is attached to the abdomen 300 of the subject, the pair of belt members 145a, 145b are pulled towards the directions of the arrows 61, B2 in the figure, respectively, by the biasing force of the protrusion spring arranged in the wind-up mechanism. In the attached state, the belt members 145a, 145b are pulled out from the reels 135a, 135b against the biasing force of the protrusion spring serving as the first biasing portion and the biasing portion with the increase in the body peripheral length of the subject when the subject performs the inhaling motion. When the subject performs the exhaling motion, the belt members 145a, 145b are wound up by the reels 135a, 135b by the biasing force of the protrusion spring serving as the first biasing portion and the biasing portion with the decrease in the body peripheral length of the subject.

According to the bioelectrical impedance measurement abdomen attachment unit 100D and the body fat measurement device 1D equipped with the same according to the present embodiment described above, the pair of belt members 145a, 145b can be constantly pulled in the attached state. Therefore, the bioelectrical impedance measurement abdomen attachment unit 100D constantly fits to the abdomen 300 of the subject even when the subject performs the breathing motion. The abdomen 300 of the subject is tightened at a substantially constant tightening strength by the bioelectrical impedance measurement abdomen attachment unit 100D based on the biasing force by the protrusion spring, whereby the plurality of electrodes 128 arranged on the electrode film 126 can be pressed against the abdomen 300 of the subject at a substantially constant load.

In the present embodiment, the bioelectrical impedance measurement abdomen attachment unit 100D to be attached to the abdomen 300 of the subject is divided to the abdomen rear surface side base portion 110D to be placed on the abdomen rear surface side of the subject and the abdomen front surface side base portion 120D to be placed on the abdomen front surface side of the subject, the portion closer to one end of the abdomen rear surface side base portion 110D and the portion closer to one end of the abdomen front surface side base portion 120D are coupled by the belt member 145a and the protrusion spring serving as the first coupling mechanism, and the portion closer to the other end of the abdomen rear surface side base portion 110D and the portion closer to the other end of the abdomen front surface side base portion 120D are coupled by the belt member 145b and the protrusion spring serving as the second coupling mechanism, whereby the bioelectrical impedance measurement abdomen attachment unit 100D can be very easily attached to the abdomen 300 of the subject. Therefore, a troublesome attachment task is unnecessary, and the bioelectrical impedance measurement abdomen attachment unit excelling in handleability and the body fat measurement device equipped with the same can be obtained.

According to the bioelectrical impedance measurement abdomen attachment unit 100D of the present embodiment, the interval between the guide unit 116a and the guide unit 116b can be adjusted using the interval adjustment mechanism according to the abdomen lateral width of the subject, and thus the bioelectrical impedance measurement abdomen attachment unit 100D can be more reliably fit to the abdomen 300 of the subject without a gap.

Furthermore, according to the bioelectrical impedance measurement abdomen attachment unit 100D and the body fat measurement device 1D equipped with the same of the present embodiment, the wrapping length of the bioelectrical impedance measurement abdomen attachment unit 100D changes following the breathing motion of the subject by appropriately adjusting the elastic force of the protrusion spring serving as the first biasing portion and the second biasing portion, and thus the subject does not feel an excessive oppressing feeling, and a bioelectrical impedance measurement abdomen attachment unit that is not painful to the subject can be obtained.

Therefore, the bioelectrical impedance measurement attachment unit which enables the electrode to be pressed against the body of the subject with a constant load in the attached state and which is not painful to the subject is realized with the bioelectrical impedance measurement abdomen attachment unit 100D according to the present embodiment. The body fat measurement device capable of calculating the body fat mass at high accuracy is realized with the body fat measurement device 1D equipped with the bioelectrical impedance measurement abdomen attachment unit 100A. Furthermore, the bioelectrical impedance measurement abdomen attachment unit and the body fat measurement device equipped with the same that enable the attachment task to be very easily performed are obtained.

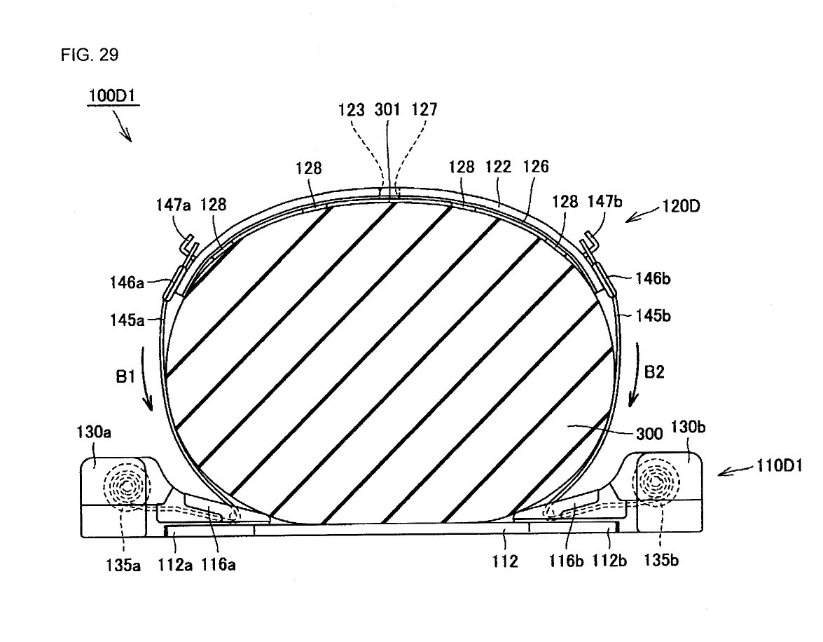

FIG. 29 is a cross-sectional view showing a state in which a bioelectrical impedance measurement abdomen attachment unit according to a variant of the present embodiment is attached to the abdomen of the subject. The configuration of a bioelectrical impedance measurement abdomen attachment unit 100D1 according to a variant of the present embodiment will now be described with reference to FIG. 29. The bioelectrical impedance measurement abdomen attachment unit 100D1 according to the present variant differs from the bioelectrical impedance measurement abdomen attachment unit 100D according to the above-described embodiment in the configuration of the abdomen rear surface side base portion 110D1.

As shown in FIG. 29, in the abdomen rear surface side base portion 110D1 of the bioelectrical impedance measurement abdomen attachment unit 100D1 according to the present variant, the wind-up unit 130a is arranged at one end 112a in the longitudinal direction of the support base 112, and the wind-up unit 130b is arranged at the other end 112b in the longitudinal direction of the support base 112. The wind-up mechanism including the reel 135a, 135b is arranged inside the wind-up unit 130a, 130b, respectively. The belt member 145a, 145b wrapped around the reel 135a, 135b is pulled out to the outside from the guide unit 116a, 116b integrally arranged with the wind-up unit 130a, 130b. The wind-up unit 130b and the guide unit 116b integrated therewith are configured to be movable with respect to the support base 112, to thereby obtain the effects similar to the effects described in the above-described embodiment.

In the first to the fourth embodiments of the present invention described above, a case in which the protrusion spring is adopted for the first and the second biasing portions has been described, but a rubber member, a constant load spring, and the like may be used in place of the protrusion spring. In particular, if the constant load spring is used, the force of winding the belt member is maintained constant regardless of the pulled-out amount of the band, and thus the abdomen of the subject is constantly tightened with a constant tightening strength by the bioelectrical impedance measurement abdomen attachment unit, whereby the electrode can always be pressed against the abdomen of the subject with a constant load.

Moreover, in the first to fourth embodiments of the present invention, a body fat measurement device in which the electrode is intended to be arranged in contact with the four limbs of the subject using the bioelectrical impedance measurement upper limb attachment unit and the lower limb attachment unit has been described, but the application of the present invention is not limited to such a body fat measurement device, and may be applied to a body fat measurement device in which the electrode is not arranged in contact with the four limbs and the electrode is intended to be arranged in contact with only the body (abdomen).

Furthermore, in the first to fourth embodiments of the present invention, the case where the present invention is applied to a body fat measurement device in which the subject is intended to take the laid position at the time of measurement and a bioelectrical impedance measurement abdomen attachment unit arranged therein has been described, but the present invention is also applicable to a body fat measurement device in which the subject is intended to take the posture other than the laid position such as a face-down position, a side position, a standing position and a sitting position, and a bioelectrical impedance measurement abdomen attachment unit arranged therein.

The embodiments disclosed herein are illustrative in all aspects and should not be construed as being restrictive. The scope of the invention is defined by the claims, and all modifications equivalent in meaning to the claims and within the scope thereof are intended to be encompassed therein.

1. A bioelectrical impedance measurement body attachment unit attached to a body of a subject to measure a bioelectrical impedance, the bioelectrical impedance measurement body attachment unit comprising:
a first base portion (110A) placed on one of a body front surface or a body rear surface of the subject in an attached state;
a second base portion (120A) placed on the other one, different from the one, of the body front surface or the body rear surface of the subject in the attached state;
a first coupling mechanism for coupling a portion closer to one end (112a) of the first base portion (110A) and a portion closer to one end (122a) of the second base portion (120A) in the attached state;
a second coupling mechanism for coupling a portion closer to the other end (112b) of the first base portion (110A) and a portion closer to the other end (122b) of the second base portion (120A) in the attached state; and
a plurality of impedance measurement electrodes (129) arranged on at least either one of the first base portion (110A) or the second base portion (120A), and brought into contact with a body surface of the subject in the attached state; wherein
the first coupling mechanism includes a first belt member (143a) having one end attached to the portion closer to the one end (112a) of the first base portion (110A) and the other end removably attached to the portion closer to the one end (122a) of the second base portion (120A), and a first biasing portion (138a) for biasing the first belt member (143a) towards the portion closer to the one end (112a) of the first base portion (110A); and
the second coupling mechanism includes a second belt member (143b) having one end attached to the portion closer to the other end (112b) of the first base portion (110A) and the other end removably attached to the portion closer to the other end (122b) of the second base portion (120A), and a second biasing portion (138b) for biasing the second belt member (143b) towards the portion closer to the other end (112b) of the first base portion (110A).

2. The bioelectrical impedance measurement body attachment unit according to claim 1, wherein
one of the first base portion (110A) or the second base portion (120A) is configured by a sheet-like member deformable so as to lie along a shape of the body surface of the subject in the attached state; and
the impedance measurement electrode (129) is arranged on a main surface on a side facing the body of the subject of the sheet-like member.

3. The bioelectrical impedance measurement body attachment unit according to claim 1, wherein
one of the first base portion (110A) or the second base portion (120A) includes a first sheet-like member deformable so as to lie along a shape of the body surface of the subject in the attached state, and a second sheet-like member (126), which is a part different from the first sheet-like member, deformable so as to lie along a shape of the body surface by being interposed between the body of the subject and the first sheet-like member in the attached state; and
the impedance measurement electrode (129) is arranged on a main surface on a side facing the body of the subject of the second sheet-like member (126).

4. The bioelectrical impedance measurement body attachment unit according to claim 1, wherein the base portion (120A) placed on the body front surface of the subject in the attached state of the first base portion (110A) and the second base portion (120A) includes a mark (123) for alignment with an umbilicus position of the subject.

5. The bioelectrical impedance measurement body attachment unit according to claim 1, wherein the first base portion (110A) includes an interval adjustment mechanism for adjusting an interval between the portion closer to the one end (112a) attached with the first coupling mechanism and the portion closer to the other end (112b) attached with the second coupling mechanism to an arbitrary distance.

6. The bioelectrical impedance measurement body attachment unit according to claim 1, wherein
the first base portion (110A) includes a first wind-up mechanism for accommodating the first belt member (143a) in a winding manner, and a second wind-up mechanism for accommodating the second belt member (143b) in a winding manner;
the first biasing portion (138a) is arranged in the first wind-up mechanism; and
the second biasing portion (138b) is arranged in the second wind-up mechanism.

7. A body fat measurement device comprising:
the bioelectrical impedance measurement body attachment unit according to claim 1;
an impedance measuring portion (12) for measuring a bioelectrical impedance of the subject using the plurality of impedance measurement electrodes (129); and
a body fat mass calculating portion (13) for calculating a body fat mass of the subject based on the bioelectrical impedance measured by the impedance measuring portion (12).

8. The body fat measurement device according to claim 7, wherein the body fat mass calculating portion (13) includes a visceral fat mass calculating part (16) for calculating a visceral fat mass of the subject.

9. The body fat measurement device according to claim 7, wherein the body fat mass calculating portion (13) includes a subcutaneous fat mass calculating part (17) for calculating a subcutaneous fat mass at an abdomen of the subject.

10. A body fat measurement device comprising:
the bioelectrical impedance measurement body attachment unit according to claim 6;
an impedance measuring portion (13) for measuring a bioelectrical impedance of the subject using the plurality of impedance measurement electrodes (129);
a body peripheral length measurement unit (30) for measuring a body peripheral length of the subject by detecting a pulled-out amount of the first belt member (143a) from the first wind-up mechanism and a pulled-out amount of the second belt member (143b) from the second wind-up mechanism; and
a body fat mass calculating portion (13) for calculating a body fat mass of the subject based on the bioelectrical impedance measured by the impedance measuring portion (12) and the body peripheral length of the subject measured by the body peripheral length measurement unit (30).

11. The body fat measurement device according to claim 10, further comprising:
a breathing state detecting portion (18) for measuring a fluctuation of the body peripheral length of the subject detected by the body peripheral length measurement unit (30) and detecting a breathing state of the subject based on the measured fluctuation of the body peripheral length of the subject with the bioelectrical impedance measurement body attachment unit attached to the body of the subject; wherein
the body fat mass calculating portion (13) calculates the body fat mass of the subject based on information of the bioelectrical impedance measured by the impedance measuring portion (12), the body peripheral length of the subject measured by the body peripheral length measurement unit (30), and the breathing state detected by the breathing state detecting portion (18).

12. The body fat measurement device according to claim 11, wherein the body fat mass calculating portion (13) extracts a bioelectrical impedance measured at a timing of transitioning from an exhaling motion to an inhaling motion detected by the breathing state detecting portion (18) from time-series data of the bioelectrical impedance measured by the impedance measuring portion (12) and calculates the body fat mass of the subject from the extracted bioelectrical impedance.

13. The body fat measurement device according to claim 10, wherein the body fat mass calculating portion (13) includes a visceral fat mass calculating part (16) for calculating a visceral fat mass of the subject.

14. The body fat measurement device according to claim 10, wherein the body fat mass calculating portion (13) includes a subcutaneous fat mass calculating part (17) for calculating a subcutaneous fat mass at an abdomen of the subject.
